SEQUENCE LISTING (8 sequences)

SEQ ID NO: 1 - protein, 20 aa, Artificial Sequence
  Chemcially Synthesized
DGIWKASFTTFTVTKYWFYR

SEQ ID NO: 2 - protein, 8 aa, Artificial Sequence
  Chemcially Synthesized
DGIWKASF

SEQ ID NO: 3 - protein, 8 aa, Artificial Sequence
  Chemcially Synthesized
SFTTFTVT

SEQ ID NO: 4 - protein, 8 aa, Artificial Sequence
  Chemcially Synthesized
VTKYWFYR

SEQ ID NO: 5 - protein, 24 aa, Artificial Sequence
  Chemcially Synthesized
  MISC_FEATURE (1)..(2): D-amino acid
  MISC_FEATURE (23)..(24): D-amino acid
KKDGIWKASFTTFTVTKYWFYRKK

SEQ ID NO: 6 - protein, 12 aa, Artificial Sequence
  Chemcially Synthesized
  MISC_FEATURE (1)..(2): D- amino acid
  MISC_FEATURE (11)..(12): D- amino acid
KKDGIWKASFKK

SEQ ID NO: 7 - protein, 12 aa, Artificial Sequence
  Chemcially Synthesized
  MISC_FEATURE (1)..(2): D- amino acid
  MISC_FEATURE (11)..(12): D- amino acid
KKSFTTFTVTKK

SEQ ID NO: 8 - protein, 12 aa, Artificial Sequence
  Chemcially Synthesized
  MISC_FEATURE (1)..(2): D- amino acid
  MISC_FEATURE (11)..(12): D- amino acid
KKVTKYWFYRKK

(19) United States
(12) Patent Application Publication
(10) Pub. No.: US 2023/0241167 A1
(43) Pub. Date: Aug. 3, 2023

(54) Use of Caveolin-1 Scaffolding Domain Peptides for Treating Disease and Disorders

(71) Applicant: MUSC Foundation for Research Development, Charleston, SC (US)

(72) Inventors: Stanley Hoffman, Charleston, SC (US); Dhandapani Kuppswamy, Mount Pleasant, SC (US); Elena Tourkina, Portland, OR (US)

(21) Appl. No.: 18/002,951

(22) PCT Filed: Jun. 29, 2021
(86) PCT No.: PCT/US21/39546

§ 371 Date: Dec. 22, 2022

Related U.S. Application Data
(60) Provisional application No. 63/046,106, filed on Jun. 30, 2020.

(51) Int. Cl. A61K 38/17; A61P 9/00; A61P 35/00; A61P 13/12

(52) U.S. Cl. CPC A61K 38/177; A61P 9/00; A61P 35/00; A61P 13/12

(57) ABSTRACT

Disclosed are CSD domain peptides and methods of use to treat diseases and disorders including fibrosis, diseases or disorders involving microvascular leakage, kidney disease, heart disease, and age-related diseases or disorders.

### CROSS REFERENCE TO RELATED APPLICATIONS

This application is a national phase application filed under 35 U.S.C. § 371 claiming benefit to International Patent Application No. PCT/US2021/039546, filed Jun. 29, 2021, which claims the benefit of priority from U.S. Provisional Application No. 63/046,106, filed Jun. 30, 2020, each of which is hereby incorporated by reference herein in its entirety.

### REFERENCE TO A “SEQUENCE LISTING”, A TABLE, OR A COMPUTER PROGRAM LISTING APPENDIX SUBMITTED AS AN ASCII TEXT FILE

The present application hereby incorporates by reference the entire contents of the text file named “206085-0085-00US_SequenceListing.txt” in ASCII format, which was created on Jun. 29, 2021, and is 2,789 bytes in size.

### STATEMENT REGARDING FEDERALLY SPONSORED RESEARCH OR DEVELOPMENT

This invention was made with government support under R01 AR062078 awarded by the National Institutes of Health and under W81XWH-11-1-0508 awarded by the Department of Defense. The government has certain rights in the invention.

### BACKGROUND OF THE INVENTION

Caveolin-1 (Cav-1) is the principal structural component of caveolae organelles in smooth muscle cells, adipocytes, fibroblasts, epithelial cells, and endothelial cells (ECs). Caveolin-1 is a master regulatory protein that binds to and thereby inhibits the function or promotes the turnover of kinases in several signaling cascades (Tourkina et al., 2005, J Biol Chem, 280:13879-13887; Couet et al., 1997, J Biol Chem, 272:6525-6533; Le Saux et al., 2008, Am J Physiol Lung Cell Mol Physiol, 295:L1007-L1017; Oka et al., 1997, J Biol Chem, 272:33416-33421; Razani et al., 2001, J Biol Chem, 276:6727-6738; Rybin et al., 1999, Circ Res, 84:980-988; Wang et al, 2008, Am J Respir Crit Care Med, 178:583-591). Caveolin-1 is underexpressed in several cell types including fibroblasts and monocytes in SSc patients and in animal models (Tourkina et al., 2005, J Biol Chem, 280:13879-13887; Lee et al., 2014, Front Pharmacol, 5:140; Lee et al., 2014, Am J Physiol Lung Cell Mol Physiol, 306:L736-L748; Del Galdo et al., 2008, Arthritis Rheum, 58:2854-2865; Kasper et al., 1998, Histochem Cell Biol, 109:41-48; Tourkina et al., 2010, Ann Rheum Dis, 69:1220-1226). This deficiency leads to Col I overexpression by fibroblasts, monocyte hypermigration toward several chemokines, and to the enhanced differentiation of monocytes into CD45+/Col F/α-smooth muscle actin+(ASMA+) fibroblastic cells (Tourkina et al., 2011, Fibrogenesis Tissue Repair, 4:15; Tourkina et al., 2005, J Biol Chem, 280:13879-13887; Reese et al., 2014, Front Pharmacol, 16:141; Lee et al., 2014, Front Pharmacol, 5:140; Tourkina et al., 2010, Ann Rheum Dis, 69:1220-1226). The effects of caveolin-1 deficiency in cells and in animals can be reversed using the caveolin-1 scaffolding domain peptide (CSD, amino acids 82-101 of caveolin-1) (Tourkina et al., 2008, Am J Physiol Lung Cell Mol Physiol, 294:L843-L861; Wang et al., 2006, J Exp Med, 203:2895-2906). CSD enters cells (Tahir et al., 2009, Cancer Biol Ther, 8:2286-2296; Tahir et al., 2008, Cancer Res, 68:731-739) which can act as a surrogate for full-length caveolin-1 by inhibiting kinases just like full-length caveolin-1 (Bucci et al., 2000, Nat Med, 6:1362-1367; Bernatchez et al., 2005, Proc Natl Acad Sci USA, 102:761-766). In addition to the profibrotic effects of low caveolin-1 and their reversal by CSD in vitro, low caveolin-1 is profibrotic in vivo. Lung, skin, and heart fibrosis are observed in caveolin-1 KO mice (DelGaldo et al., 2008, Arthritis Rheum, 58:2854-65; Cohen et al., 2003, Amer Jour of Cell Phys, 284:C457-74; Drab et al., 2001, Science, 293:2449-52; Razani et al., 2001, J Biol Chem, 276:38121-38).). CSD also inhibits fibrosis in vivo in lung, skin, and heart (Tourkina et al., 2011, Fibrogenesis Tissue Repair, 4:15; Reese et al, 2014, Frontiers in Pharma, 5: epub; Tourkina et al., 2008, Amer Journal of Lung Cell Mol Phys, 294:L843-61; Pleasant-Jenkins et al, 2017, Lab Invest, 97:370-382). In contrast, in endothelial cells (that express caveolin-1 at high levels), in some cases the beneficial effects of CSD may result from it acting as a competitor to the function of caveolin-1 (Chidlow et al., 2009, Gastroenterology, 136(2):575-84 e2; Tahir et al., 2009, Cancer biology & therapy, 8(23):2286-96).

There remains a need in the art for compositions and methods for treating or preventing diseases and disorders including fibrosis, microvascular leakage, and aging and aging-related diseases and disorders. The present invention satisfies this unmet need.

### SUMMARY OF THE INVENTION

In one embodiment, the invention relates to a method of treating or preventing microvascular leakage or a disease or disorder associated therewith, kidney disease, heart disease, or an age-related disease or disorder in a subject, the method comprising administering to a subject in need thereof an effective amount of a composition comprising a CSD domain peptide, or fragment or variant thereof, or a nucleic acid molecule encoding a CSD domain peptide, or fragment or variant thereof, wherein the CSD domain peptide comprises an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7 and SEQ ID NO:8.

In one embodiment, the disease or disorder is an age-related disease or disorder. In one embodiment, the disease or disorder is atherosclerosis, cardiovascular disease, microvascular leakage, cancer, arthritis, cataracts, osteoporosis, Alzheimer's disease and related neurodegenerative diseases or hypertension.

In one embodiment, the disease or disorder is a kidney disease. In one embodiment, the kidney disease is renal inflammatory injury, kidney dysfunction, chronic kidney failure, or hypertension.

In one embodiment, the disease or disorder is a heart disease. In one embodiment, the heart disease is cardiac hypertrophy, atherosclerosis, cardiomyopathy, stroke, or hypertension.

In one embodiment, the disease or disorder is associated with microvascular leakage. In one embodiment, the disease or disorder is congestive heart failure, scleroderma and interstitial lung diseases in general, asthma, kidney failure, neurodegenerative diseases including Alzheimer's disease and vascular dementia, cancer, venous thrombosis, diabetes and complications of diabetes, sepsis, or acute respiratory distress syndrome (ARDS).

In one embodiment, the invention relates to a method of treating or preventing a disease or disorder in a subject, the method comprising administering to a subject in need thereof an effective amount of a composition comprising a CSD domain peptide, or fragment or variant thereof, or a nucleic acid molecule encoding a CSD domain peptide, or fragment or variant thereof, wherein the CSD domain peptide comprises an amino acid sequence selected from the group consisting of SEQ ID NO:5, SEQ ID NO:6, SEQ ID NO:7 and SEQ ID NO:8.

In one embodiment, the disease or disorder is an age-related disease or disorder selected from the group consisting of atherosclerosis, cardiovascular disease, microvascular leakage, cancer, arthritis, cataracts, osteoporosis, Alzheimer's disease and related neurodegenerative diseases and hypertension.

In one embodiment, the disease or disorder is fibrosis or a fibrosis-related disease or disorder.

In one embodiment, the disease or disorder is microvascular leakage or a microvascular-leakage related disease or disorder. In one embodiment, the disease or disorder is congestive heart failure, scleroderma and interstitial lung diseases in general, asthma, kidney failure, neurodegenerative diseases including Alzheimer's disease and vascular dementia, cancer, venous thrombosis, diabetes and complications of diabetes, sepsis, or acute respiratory distress syndrome (ARDS).

In one embodiment, the disease or disorder is kidney disease. In one embodiment, the kidney disease is renal inflammatory injury, kidney dysfunction, chronic kidney failure, or hypertension.

In one embodiment, the disease or disorder is heart disease. In one embodiment, the heart disease is cardiac hypertrophy, atherosclerosis, cardiomyopathy, stroke, or hypertension.

In one embodiment, the invention relates to a modified CSD domain peptide comprising an amino acid sequence selected from the group consisting of SEQ ID NO:5, SEQ ID NO:6, SEQ ID NO:7 and SEQ ID NO:8, or a fragment or variant thereof.

In one embodiment, the invention relates to a composition comprising a modified CSD domain peptide comprising an amino acid sequence selected from the group consisting of SEQ ID NO:5, SEQ ID NO:6, SEQ ID NO:7 and SEQ ID NO:8, or a fragment or variant thereof. In one embodiment, the composition further comprises a pharmaceutically acceptable carrier.

In one embodiment, the composition treats or prevents a disease or disorder in a subject. In one embodiment, the disease or disorder is an age-related disease or disorder selected from the group consisting of atherosclerosis, cardiovascular disease, microvascular leakage, cancer, arthritis, cataracts, osteoporosis, Alzheimer's disease and related neurodegenerative diseases and hypertension. In one embodiment, the disease or disorder is fibrosis or a fibrosis-related disease or disorder. In one embodiment, the disease or disorder is microvascular leakage or a microvascular-leakage related disease or disorder. In one embodiment, the disease or disorder is congestive heart failure, scleroderma and interstitial lung diseases in general, asthma, kidney failure, neurodegenerative diseases including Alzheimer's disease and vascular dementia, cancer, venous thrombosis, diabetes and complications of diabetes, sepsis, or acute respiratory distress syndrome (ARDS). In one embodiment, the disease or disorder is kidney disease. In one embodiment, the kidney disease is renal inflammatory injury, kidney dysfunction, chronic kidney failure, tumor growth and metastasis or hypertension. In one embodiment, the disease or disorder is heart disease. In one embodiment, the heart disease is cardiac hypertrophy, atherosclerosis, cardiomyopathy, stroke, or hypertension.

In one embodiment, the composition is formulated for administration by a delivery route selected from the group consisting of intranasal, oro-pharyngeal and intraperitoneal.

In one embodiment, the invention relates to a composition for treating or preventing microvascular leakage or a disease or disorder associated therewith, kidney disease, heart disease, or an age-related disease or disorder, comprising a CSD domain peptide comprising an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7 and SEQ ID NO:8, or a fragment or variant thereof.

In one embodiment, the disease or disorder is an age-related disease or disorder. In one embodiment, the disease or disorder is atherosclerosis, cardiovascular disease, kidney disease, microvascular leakage, cancer, arthritis, cataracts, osteoporosis, AD and related neurodegenerative diseases, complications of diabetes, or hypertension.

In one embodiment, the disease or disorder is a kidney disease. In one embodiment, the kidney disease is renal inflammatory injury, kidney dysfunction, chronic kidney failure, or hypertension.

In one embodiment, the disease or disorder is a heart disease. In one embodiment, the heart disease is cardiac hypertrophy, atherosclerosis, cardiomyopathy, stroke, or hypertension.

In one embodiment, the disease or disorder is a microvascular leakage associated disease or disorder. In one embodiment, the microvascular leakage associated disease or disorder is congestive heart failure, scleroderma and interstitial lung diseases in general, asthma, kidney failure, neurodegenerative diseases including Alzheimer's disease and vascular dementia, cancer, venous thrombosis, diabetes and complications of diabetes, sepsis, or acute respiratory distress syndrome (ARDS).

In one embodiment, the composition is formulated for administration by a delivery route selected from the group consisting of intranasal, oro-pharyngeal and intraperitoneal.

### BRIEF DESCRIPTION OF THE DRAWINGS

The following description of exemplary embodiments of the invention will be better understood when read in conjunction with the appended drawings. It should be understood, that the invention is not limited to the precise arrangements and instrumentalities of the embodiments shown in the drawings.

FIG. 1 provides an overview of an experiment using Full-Length CSD, 82-89, 88-95, and 94-101 (SEQ ID NO:1, SEQ ID NO:2, SEQ ID NO:3, and SEQ ID NO:4 respectively) in a congestive heart failure (CHF) model.

FIG. 2 depicts exemplary experimental data demonstrating that CSDs suppress the effects of AngII on HW/BW ratio, left ventricle (LV) mass and posterior wall thickness (pWTh-d) in a CHF model.

FIG. 3 depicts exemplary experimental data demonstrating that CSDs suppress the effects of AngII on Ejection Fraction (EF), Fractional Shortening (FS), and Isovolumic Relaxation Time (IVRT) in a CHF model.

FIG. 4 depicts exemplary experimental data demonstrating that CSD subdomains suppress the effects of AngII-induced fibrosis in the heart, as measured in terms of increased Col I deposition and HSP47 level.

FIG. 5 depicts exemplary experimental data demonstrating that AngII induces microvascular leakage in the heart (measured in terms of IgG heavy chain level in the tissue) that is almost completely suppressed both by 82-89 and 88-95

FIG. 6 provides an overview of an experiment using W82-89 (SEQ ID NO: 6) in a CHF model.

FIG. 7 depicts exemplary experimental data demonstrating that W82-89 suppresses AngII-induced pathological increases in HW/BW ratio, microvascular leakage, and Col I levels in the heart.

FIG. 8 depicts a summary of the effective domains.

FIG. 9 depicts the experimental design for experiments to demonstrate that CSD suppresses aging-associated pathological changes in the heart and kidney.

FIG. 10 depicts exemplary experimental data demonstrating that CSD reverses effects of aging on fibrosis and microvascular leakage in the heart.

FIG. 11 depicts exemplary experimental data demonstrating that CSD reverses effects of aging on fibrosis and microvascular leakage in the kidney.

FIG. 12 depicts exemplary experimental data demonstrating representative examples of picrosirius red staining of heart and kidney tissue sections from young or aged mice with and without CSD treatment.

FIG. 13 depicts exemplary experimental data demonstrating the reversal of the effects of aging in the heart and kidney by CSD.

FIG. 14 depicts exemplary experimental data demonstrating that the CSD had a positive effect on fractional shortening (FS) and ejection fraction (EF), and isovolumic relaxation time (IVRT) in the aged heart.

FIG. 15 depicts exemplary experimental data demonstrating that cardiomyocyte hypertrophy was increased in aged mice and this increase was reversed by CSD.

FIG. 16 provides an overview of an experiment using CSD (SEQ ID NO: 1) to explore the effects of CSD on aging in the brain.

FIG. 17 depicts exemplary experimental data demonstrating that 18-month old mice have much higher levels of microvascular leakage and fibrosis in the brain than young mice, and that systemic CSD treatment decreases these levels almost to the levels observed in healthy 3-month old mice.

FIG. 18 depicts exemplary experimental data demonstrating that 18-month old mice have much higher levels of activated tyrosine kinases in the brain than young mice, and that systemic CSD treatment decreases these levels almost to the levels observed in healthy 3-month old mice.

FIG. 19 depicts the experimental design for experiments demonstrating suppression of lung and skin fibrosis by unmodifed CSD subdomains.

FIG. 20 depicts exemplary experimental data demonstrating the suppression of lung fibrosis by CSD and unmodified subdomains. (Upper) Masson's Trichrome-stained lung tissue sections demonstrate the massive fibrosis caused by bleomycin and its suppression by 82-89. (Lower) Ashcroft scores were determined by a veterinary pathologist blinded to the identity of the samples (n=6 per group). * p<0.05 for Bleo+treatment vs Bleo+vehicle.

FIG. 21 depicts data demonstrating the suppression of dermal fibrosis and loss of intradermal fat by CSD and CSD subdomain peptides. Skin in the vicinity of the pump outlet was harvested for measurement of the thickness of the dermis and the intradermal fat. \hat{ }\hat{ }\hat{ }p<0.001 for Saline/Vehicle vs Bleo/Vehicle. ***p<0.001, **p<0.01, *p<0.05 for Bleo/Vehicle vs Bleo/Peptide Treatments.

FIG. 22 depicts data demonstrating the suppression of monocyte migration by CSD and unmodified subdomains.

FIG. 23 depicts the experimental design for experiments demonstrating suppression of lung and skin fibrosis by a modified, water-soluble version of CSD (WCSD).

FIG. 24 depicts survival and histology data demonstrating suppression of lung and skin fibrosis by a modified, water-soluble CSD. The lower panels are Masson's Trichrome-stained tissue sections.

FIG. 25 depicts exemplary experimental data demonstrating that WCSD suppresses bleomycin-induced lung fibrosis through its effects on fibrocytes, ECM proteins, myofibroblast markers, and microvascular leakage.

FIG. 26 depicts data demonstrating the inhibition of tumor growth by WCSD.

FIG. 27 depicts data demonstrating that nintedanib and modified, water-soluble versions of CSD have distinct kinase inhibition profiles.

FIG. 28 depicts data demonstrating that modified, water-soluble versions of CSD are more active as kinase inhibitors than their parental, unmodified forms.

FIG. 29 depicts data demonstrating that intranasal (i.n.) is a promising route of delivery for modified, water-soluble versions of CSD.

FIG. 30 depicts data demonstrating the uptake of fluorescent peptides by primary mouse lung fibroblast cultures.

FIG. 31 depicts data demonstrating the plasma levels of W82-89 following different routes of administration.

FIG. 32 depicts data demonstrating that W82-89 uptake into plasma following i.p. administration is much more effective than uptake of CSD or WCSD.

### DETAILED DESCRIPTION

The present invention is based partly on the experiments demonstrating that CSD domain peptides are effective in suppressing fibrosis and microvascular leakage and other aspects of congestive heart failure (CHF) and kidney disease induced by angiotensin II (AngII) Further, the CSD domain peptides were effective in treating aging-associated pathological changes in the heart, kidney, and brain. Therefore, in some embodiments, the invention relates to methods of treating fibrosis, microvascular leakage and diseases and disorders associated therewith, and aging and aging-related diseases and disorders, heart disease, and kidney disease in a subject comprising administering the CSD domain peptides of the invention.

### Definitions

Unless defined otherwise, all technical and scientific terms used herein have the same meaning as commonly understood by one of ordinary skill in the art to which this invention belongs. Any methods and materials similar or equivalent to those described herein can be used in the practice or testing of the present invention.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, ±0.1%, less than ±0.1%, or any percentage therebetween from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

There term “in combination with” is used herein to that the indicated treatments are administered concurrently or that a first treatment is administered sequentially with one or more additional treatment.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

The term “fusion protein” used herein refers to two or more peptides, polypeptides, or proteins operably linked to each other.

“Nucleic acid” or “oligonucleotide” or “polynucleotide” or grammatical equivalents used herein means at least two nucleotides covalently linked together. The term “nucleic acid” includes single-, double-, or multiple-stranded DNA, RNA and analogs (derivatives) thereof. Oligonucleotides are typically from about 5, 6, 7, 8, 9, 10, 12, 15, 25, 30, 40, 50 or more nucleotides in length, up to about 100 nucleotides in length. Nucleic acids and polynucleotides are a polymers of any length, including longer lengths, e.g., 200, 300, 500, 1000, 2000, 3000, 5000, 7000, 10,000, etc. In certain embodiments, the nucleic acids herein contain phosphodiester bonds. In other embodiments, nucleic acid analogs are included that may have alternate backbones, comprising, e.g., phosphoramidate, phosphorothioate, phosphorodithioate, or O-methylphosphoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made.

A nucleotide sequence is “operably linked” when it is placed into a functional relationship with another nucleotide sequence. For example, a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

Unless otherwise specified, a “nucleotide sequence encoding an amino acid sequence” includes all nucleotide sequences that are degenerate versions of each other and that encode the same amino acid sequence.

Twenty amino acids are commonly found in proteins. Those amino acids can be grouped into nine classes or groups based on the chemical properties of their side chains. Substitution of one amino acid residue for another within the same class or group is referred to herein as a “conservative” substitution. Conservative amino acid substitutions can frequently be made in a protein without significantly altering the conformation or function of the protein. Substitution of one amino acid residue for another from a different class or group is referred to herein as a “non-conservative” substitution. In contrast, non-conservative amino acid substitutions tend to modify conformation and function of a protein.

In some embodiments, the conservative amino acid substitution comprises substituting any of glycine (G), alanine (A), isoleucine (I), valine (V), and leucine (L) for any other of these aliphatic amino acids; serine (S) for threonine (T) and vice versa; aspartic acid (D) for glutamic acid (E) and vice versa; glutamine (Q) for asparagine (N) and vice versa; lysine (K) for arginine (R) and vice versa; phenylalanine (F), tyrosine (Y) and tryptophan (W) for any other of these aromatic amino acids; and methionine (M) for cysteine (C) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pKs of these two amino acid residues are not significant. Still other changes can be considered “conservative” in particular environments (see, e.g., BIOCHEMISTRY at pp. 13-15, 2nd ed. Lubert Stryer ed. (Stanford University); Henikoff et al, Proc. Nat'l Acad. Set USA (1992) 89: 10915-10919; Lei et al., J. Biol. Chem. (1995) 270(20): 1 1882-1 1886).

In some embodiments, the non-conservative amino acid substitution comprises substituting any of glycine (G), alanine (A), isoleucine (I), valine (V), and leucine (L) for any of serine (S), threonine (T), aspartic acid (D), glutamic acid (E), glutamine (Q), asparagine (N), lysine (K), arginine (R), phenylalanine (F), tyrosine (Y), tryptophan (W), methionine (M), cysteine (C), histidine (H), and proline (P). In some embodiments, the non-conservative amino acid substitution comprises substituting any of serine (S) and threonine (T) for any of glycine (G), alanine (A), isoleucine (I), valine (V), leucine (L), aspartic acid (D), glutamic acid (E), glutamine (Q), asparagine (N), lysine (K), arginine (R), phenylalanine (F), tyrosine (Y), tryptophan (W), methionine (M), cysteine (C), histidine (H) and proline (P). In some embodiments, the non-conservative amino acid substitution comprises substituting any of aspartic acid (D) and glutamic acid (E) for any of glycine (G), alanine (A), isoleucine (I), valine (V), leucine (L), serine (S), threonine (T), glutamine (Q), asparagine (N), lysine (K), arginine (R), phenylalanine (F), tyrosine (Y), tryptophan (W), methionine (M), cysteine (C), histidine (H), and proline (P). In some embodiments, the non-conservative amino acid substitution comprises substituting any of glutamine (Q) and asparagine (N) for any of glycine (G), alanine (A), isoleucine (I), valine (V), leucine (L), serine (S), threonine (T), aspartic acid (D), glutamic acid (E), lysine (K), arginine (R), phenylalanine (F), tyrosine (Y), tryptophan (W), methionine (M), cysteine (C), histidine (H), and proline (P). In some embodiments, the non-conservative amino acid substitution comprises substituting any of lysine (K) and arginine (R) for any of glycine (G), alanine (A), isoleucine (I), valine (V), leucine (L), serine (S), threonine (T), aspartic acid (D), glutamic acid (E), glutamine (Q), asparagine (N), phenylalanine (F), tyrosine (Y), tryptophan (W), methionine (M), cysteine (C), histidine (H), and proline (P). In some embodiments, the non-conservative amino acid substitution comprises substituting any of phenylalanine (F), tyrosine (Y), and tryptophan (W) for any of glycine (G), alanine (A), isoleucine (I), valine (V), leucine (L), serine (S), threonine (T), aspartic acid (D), glutamic acid (E), glutamine (Q), asparagine (N), lysine (K), arginine (R), methionine (M), cysteine (C), histidine (H), and proline (P). In some embodiments, the non-conservative amino acid substitution comprises substituting any of methionine (M) and cysteine (C) for any of glycine (G), alanine (A), isoleucine (I), valine (V), leucine (L), serine (S), threonine (T), aspartic acid (D), glutamic acid (E), glutamine (Q), asparagine (N), lysine (K), arginine (R), phenylalanine (F), tyrosine (Y), tryptophan (W), histidine (H), and proline (P). In some embodiments, the non-conservative amino acid substitution comprises substituting histidine (H) for any of glycine (G), alanine (A), isoleucine (I), valine (V), leucine (L), serine (S), threonine (T), aspartic acid (D), glutamic acid (E), glutamine (Q), asparagine (N), lysine (K), arginine (R), phenylalanine (F), tyrosine (Y), tryptophan (W), methionine (M), cysteine (C), and proline (P). In some embodiments, the non-conservative amino acid substitution comprises substituting proline (P) for any of glycine (G), alanine (A), isoleucine (I), valine (V), leucine (L), serine (S), threonine (T), aspartic acid (D), glutamic acid (E), glutamine (Q), asparagine (N), lysine (K), arginine (R), phenylalanine (F), tyrosine (Y), tryptophan (W), methionine (M), cysteine (C), and histidine (H).

“Polypeptide,” “peptide,” and “protein” are used herein interchangeably and mean any peptide-linked chain of amino acids, regardless of length or post-translational modification. As noted below, the polypeptides described herein can be, e.g., wild-type proteins, biologically-active fragments of the wild-type proteins, or variants of the wild-type proteins or fragments. Variants, in accordance with the disclosure, can contain amino acid substitutions, deletions, or insertions. The substitutions can be conservative or non-conservative. In some embodiments, conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine.

Following expression, the proteins (e.g. CSD domain peptides) can be isolated. The term “purified” or “isolated” as applied to any of the proteins described herein (e.g., a conjugate described herein, antibody or antigen-binding fragment thereof described herein) refers to a polypeptide that has been separated or purified from components (e.g., proteins or other naturally-occurring biological or organic molecules) which naturally accompany it, e.g., other proteins, lipids, and nucleic acid in a prokaryote expressing the proteins. Typically, a polypeptide is purified when it constitutes at least 60 (e.g., at least 65, 70, 75, 80, 85, 90, 92, 95, 97, or 99) %, by weight, of the total protein in a sample.

A “label” or a “detectable moiety” is a composition detectable by spectroscopic, photochemical, biochemical, immunochemical, chemical, magnetic resonance imaging, or other physical means. For example, useful detectable moieties include 32P, fluorescent dyes, electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, standard superparamagnetic iron oxide (“SSPIO”), SSPIO nanoparticle aggregates, polydisperse superparamagnetic iron oxide (“PSPIO”), PSPIO nanoparticle aggregates, monochrystalline SPIO, monochrystalline SPIO aggregates, monochrystalline iron oxide nanoparticles, monochrystalline iron oxide, other nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. Detectable moieties also include any of the above compositions encapsulated in nanoparticles, particles, aggregates, coated with additional compositions, derivatized for binding to a targeting agent (e.g. antibody or antigen binding fragment). Any method known in the art for conjugating an antibody to the label may be employed, e.g., using methods described in Hermanson, Bioconjugate Techniques 1996, Academic Press, Inc., San Diego.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration. The term “diagnostically acceptable” is used synonymously with “physiologically acceptable” and “pharmacologically acceptable” and refers to diagnostic compositions.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

Treatment,” “treat,” or “treating” mean a method of reducing the effects of a disease or condition. Treatment can also refer to a method of reducing the disease or condition itself rather than just the symptoms. The treatment can be any reduction from native levels and can be but is not limited to the complete ablation of the disease, condition, or the symptoms of the disease or condition. Therefore, in the disclosed methods, “treatment” can refer to a 10%, 20%, 30%, 40%, 50%, 60%, 70%, 80%, 90%, or 100% reduction in the severity of an established disease or the disease progression. For example, a disclosed method for reducing the effects of a disease or disorder is considered to be a treatment if there is a 10% reduction in one or more symptoms of the disease in a subject with the disease when compared to native levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition.

As used herein, the terms “treat” and “prevent” may refer to any delay in onset, reduction in the frequency or severity of symptoms, amelioration of symptoms, improvement in patient comfort or function (e.g. joint function), decrease in severity of the disease state, etc. The effect of treatment can be compared to an individual or pool of individuals not receiving a given treatment, or to the same patient prior to, or after cessation of, treatment. The term “prevent” generally refers to a decrease in the occurrence of a given disease (e.g. an autoimmune, inflammatory autoimmune, cancer, infectious, immune, or other disease) or disease symptoms in a patient. As indicated above, the prevention may be complete (no detectable symptoms) or partial, such that fewer symptoms are observed than would likely occur absent treatment.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The endothelium is directly involved in many diseases and disorders including, but not limited to, diseases and disorders involving microvascular leakage, peripheral vascular disease, stroke, heart disease, diabetes, insulin resistance, chronic kidney failure, tumor growth and metastasis, venous thrombosis, asthma, retinopathy and other complications of diabetes, ARDS (for example, induced by viral infection or by lung injury), sepsis and severe viral infectious diseases. Further, endothelial dysfunction has been implicated in the development of neurodegenerative conditions, such as Alzheimer's disease (AD).

The invention is based, in part, on the discovery that administration of CSD domain peptides was able to inhibit microvascular leakage, and suppress the pathological effects of AngII and bleomycin and of aging.

Accordingly, the compositions of the present invention can be used to treat fibrosis, microvascular leakage, aging and aging-related diseases and disorders, heart disease, and kidney disease.

In various embodiments, the compositions and methods of the invention can be used to treat a fibrotic disease or disorder. Fibrosis can occur in many tissues within the body, including, but not limited to, lungs, liver, heart, kidney, brain, joints, skin, and bone marrow. Non-limiting examples of fibrotic diseases and disorders that can treated using the compositions and methods described herein include, but are not limited to interstitial lung disease, idiopathic pulmonary fibrosis, lung fibrosis, asthma, chronic obstructive pulmonary disease (COPD), Raynaud's phenomenon, pulmonary fibrosis, cirrhosis, atrial fibrosis, endomyocardial fibrosis, arthrofibrosis, Crohn's Disease, mediastinal fibrosis, myelofibrosis, tubulointerstitial fibrosis, hepatic fibrosis, premacular fibrosis, retinal fibrosis, dermal fibrosis, wound-associated fibrosis, Peyronie's disease, nephrogenic systemic fibrosis, progressive massive fibrosis, retroperitoneal fibrosis, fibroma, scleroderma, and radiation-induced fibrosis especially due to radiation therapy.

Non-limiting examples of aging-related diseases and disorders that can be treated using the compositions and methods described herein include, but are not limited to, atherosclerosis, cardiovascular disease, kidney disease, microvascular leakage, cancer, arthritis, cataracts, osteoporosis, AD and related neurodegenerative diseases, complications of diabetes, and hypertension.

Non-limiting examples of diseases and disorders involving microvascular leakage that can treated using the compositions and methods described herein include, but are not limited to congestive heart failure, scleroderma and interstitial lung diseases in general, asthma, kidney failure, neurodegenerative diseases including Alzheimer's disease and vascular dementia, cancer, venous thrombosis, diabetes and complications of diabetes, sepsis, and acute respiratory distress syndrome (ARDS).

Non-limiting examples of heart and kidney diseases and disorders that can be treated using the compositions and methods described herein include, but are not limited to, cardiac hypertrophy, atherosclerosis, cardiomyopathy, stroke, renal inflammatory injury, kidney dysfunction, chronic kidney failure, and hypertension.

It is understood by those of skill in the art that the term treating, as used herein, includes repairing, replacing, augmenting, improving, preventing occurrence or recurrence, rescuing, repopulating, or regenerating.

Caveolin-1 Scaffolding Domain Peptide (CSD)

In one embodiment, the method comprises administration of a CSD domain peptide to a subject in need thereof for the treatment of a disease or disorder.

Caveolin-1 is the principal coat protein of caveolae. Caveolae were originally observed in electron microscopic images as flask-shaped invaginations in the plasma membrane. These cholesterol- and sphingolipid-rich organelles function in endocytosis, vesicular trafficking, and in the compartmentalization of specific signaling cascades. The caveolin family of caveolae coat proteins contains three members of which caveolin-1 and -2 are abundantly expressed in adipocytes, endothelial cells, and fibroblasts. Caveolins serve as scaffolds for signaling molecules including members of the MAP kinase family, isoforms of PKC, Akt, G proteins, Src-family kinases, and growth factor receptors. The ability of caveolin-1 to bind to a variety of kinases and thereby inhibit their activity has been mapped to a sequence known as the caveolin-1 scaffolding domain (CSD, amino acids 82-101 of caveolin-1; DGIWKASFTTFTVTKYWFYR (SEQ ID NO:1).

In one embodiment, a subdomain of the CSD domain peptide comprises at least six consecutive amino acid residues of the CSD domain peptide. In one embodiment, the subdomain comprises an amino acid sequence of DGIWKASF (SEQ ID NO:2), SFTTFTVT (SEQ ID NO:3), or VTKYWFYR (SEQ ID NO:4).

In one embodiment, the CSD domain peptide further comprises at least one additional amino acid residue. In various embodiments, the at least one additional amino acid residue modifies the peptide to: 1) increase the water-solubility of the peptide, 2) protect the peptides against proteolysis by exoproteases, 3) carry peptides across the plasma membrane that would otherwise not cross the plasma membrane or any combination thereof.

In one embodiment, the CSD domain peptide further comprises at least 1, 2, 3, 4, 5 or more than 5 D-Lysine residue at the C-terminal or N-terminal ends of the peptide. In one embodiment, the CSD domain peptide further comprises at least 1, 2, 3, 4, 5 or more than 5 D-Lysine residue at each of the C-terminal and N-terminal ends of the peptide. In one embodiment, the CSD domain peptide further comprises 2 D-Lysine residues at the C-terminus and 2 D-Lysine residues (k) at the N-terminus. In one embodiment, the subdomain comprises an amino acid sequence of kkDGIWKASFTTFTVTKYWFYRkk (SEQ ID NO:5), kkDGIWKASFkk (SEQ ID NO:6), kkSFTTFTVTkk (SEQ ID NO:7), or kkVTKYWFYRkk (SEQ ID NO:8).

In one embodiment, the CSD domain peptide further comprises at least one protein modification. In one embodiment, the CSD domain peptide comprises at least one of an N-terminal acetylation, and a C-terminal amide.

It is understood and herein contemplated that there are a number of variations of the CSD, or a subdomain thereof, that can be used in the disclosed methods of treatment. Specifically contemplated herein are modifications or mutations made to the CSD, or a subdomain thereof, that do not inhibit target binding, but can aid the peptide in, for example, avoiding proteolysis. Modifications and mutations of the CSD, or a subdomain thereof, that can be made include those described in detail in U.S. Pat. No. 8,058,227 B2 which is incorporated herein in its entirety.

It is further understood that the CSD domain peptide, or subdomain thereof, may be modified to aid entry into a cell. Therefore, contemplated herein are any known modifications that can be made to the CSD, or a subdomain thereof, that can aid entry into a cell. Also contemplated here are modifications to CSD or a subdomain thereof that, based on empirical data, aid entry into a cell. In addition, contemplated herein are methods of treating fibrosis, microvascular leakage, aging and aging-related diseases and disorders, heart disease, and kidney disease comprising contacting a subject, with a composition comprising a fusion peptide comprising a CSD, or a subdomain thereof.

It is understood and herein contemplated that CSD domain peptides treat fibrosis, microvascular leakage, aging and aging-related diseases and disorders, heart disease, and kidney disease through the binding to caveolin-1 binding domains. It is further understood that any variants of CSD such as derivatives or analogues of CSD or agents capable of binding to a caveolin-1 target will also be effective in the treatment of fibrosis, microvascular leakage, aging and aging-related diseases and disorders, heart disease, and kidney disease. The identity of such agents, analogues, or derivatives of CSD, or a subdomain thereof, can be determined by their beneficial effects on disease models in vivo and in vitro as compared to other versions of CSD.

The invention should also be construed to include any form of a peptide variant having substantial homology to an amino acid sequence disclosed herein. In one embodiment, a peptide variant is at least about 50%, 70%, 80%, 85%, 86%, 87%, 88%, 89%, 90%, 91%, 92%, 93%, 94%, 95%, 96%, 97%, 98%, or 99% homologous to an amino acid sequence disclosed herein.

The invention should also be construed to include any form of a fragment having a substantial length of an amino acid sequence disclosed herein. In one embodiment, a fragment is at least about 50%, 70%, 80%, 85%, 86%, 87%, 88%, 89%, 90%, 91%, 92%, 93%, 94%, 95%, 96%, 97%, 98%, or 99% of the length of an amino acid sequence disclosed herein.

The invention should also be construed to include any form of a fragment of a peptide variant, having both substantial homology to and a substantial length of an amino acid sequence disclosed herein. In one embodiment, a fragment of a peptide variant is at least about 50%, 70%, 80%, 85%, 86%, 87%, 88%, 89%, 90%, 91%, 92%, 93%, 94%, 95%, 96%, 97%, 98%, or 99% homologous to an amino acid sequence disclosed herein, and is at least about 50%, 70%, 80%, 85%, 86%, 87%, 88%, 89%, 90%, 91%, 92%, 93%, 94%, 95%, 96%, 97%, 98%, or 99% of the length of an amino acid sequence disclosed herein.

The peptide may alternatively be made by recombinant means or by cleavage from a longer peptide. The peptide may be confirmed by amino acid analysis or sequencing.

The variants of the peptides according to the present invention may be (i) one in which one or more of the amino acid residues are substituted with a conserved or non-conserved amino acid residue (e.g., a conserved amino acid residue) and such substituted amino acid residue may or may not be one encoded by the genetic code, (ii) one in which there are one or more modified amino acid residues, e.g., residues that are modified by the attachment of substituent groups, (iii) fragments of the peptides or domains described herein and/or (iv) one in which the peptide is fused with another peptide or peptide, such as a leader or secretory sequence or a sequence which is employed for purification (for example, His-tag) or for detection (for example, Sv5 epitope tag). The fragments include peptides or peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two peptides is determined by comparing the amino acid sequence and its conserved amino acid substitutes of one polypeptide to a sequence of a second polypeptide. Variants are defined to include peptide sequences different from the original sequence, e.g., different from the original sequence in less than 40% of residues per segment of interest, different from the original sequence in less than 25% of residues per segment of interest, different by less than 10% of residues per segment of interest, or different from the original peptide sequence in just a few residues per segment of interest and at the same time sufficiently homologous to the original sequence to preserve the functionality of the original sequence. The present invention includes amino acid sequences that are at least 60%, 65%, 70%, 72%, 74%, 76%, 78%, 80%, 90%, or 95% similar or identical to the original amino acid sequence. The degree of identity between two polypeptides may be determined using computer algorithms and methods that are widely known for the persons skilled in the art. The identity between two amino acid sequences may be determined by using the BLASTP algorithm (BLAST Manual, Altschul, S., et al., NCBI NLM NIH Bethesda, Md. 20894, Altschul, S., et al., J. Mol. Biol. 215: 403-410 (1990)).

The peptide of the invention may or may not be post-translationally modified. For example, post-translational modifications that fall within the scope of the present invention include signal peptide cleavage, glycosylation, acetylation, isoprenylation, proteolysis, myristoylation, peptide folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction. A polypeptide or peptide of the invention may be phosphorylated using conventional methods such as the method described in Reedijk et al. (The EMBO Journal 11(4):1365, 1992).

The peptide of the invention may include unnatural amino acids formed by post-translational modification or by introducing unnatural amino acids during translation. A variety of approaches are available for introducing unnatural amino acids during polypeptide translation.

A peptide of the invention may be conjugated with other molecules, such as polyethylene glycol (PEG). This may be accomplished by inserting cysteine mutations or unnatural amino acids that can be modified with a chemically reactive PEG derivative. In one embodiment, the peptide is conjugated to other peptides, to prepare fusion peptides. This may be accomplished, for example, by the synthesis of N-terminal or C-terminal fusion peptides provided that the resulting fusion peptide retains the functionality of the peptide described herein.

Cyclic derivatives of the peptides of the invention are also part of the present invention. Cyclization may allow the peptide to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-comprising amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic peptide which is more flexible than the cyclic peptides comprising peptide bond linkages as described above. A more flexible peptide may be prepared by introducing cysteines at the right and left position of the peptide and forming a disulfide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

The invention also relates to a peptide described herein fused to, or integrated into, a targeting protein, or a targeting domain capable of directing the resulting protein to a desired cellular component or cell type or tissue. The chimeric or fusion proteins may also contain additional amino acid sequences or domains. The chimeric or fusion proteins are recombinant in the sense that the various components are from different sources, and as such are not found together in nature (i.e., are heterologous).

In one embodiment, the targeting domain can be a membrane spanning domain, a membrane binding domain, or a sequence directing the protein to associate, for example, with vesicles or with the cell surface. In one embodiment, the targeting domain can target a protein to a particular cell type or tissue. For example, the targeting domain can be a cell surface ligand or an antibody against cell surface antigens of a target tissue. A targeting domain may target a protein of the invention to a cellular component.

A protein of the invention may be synthesized by conventional techniques. For example, the proteins may be synthesized by chemical synthesis using solid phase peptide synthesis. These methods employ either solid or solution phase synthesis methods (see for example, J. M. Stewart, and J. D. Young, Solid Phase Peptide Synthesis, 2nd Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis). By way of example, a polypeptide of the invention may be synthesized using 9-fluorenyl methoxycarbonyl (Fmoc) solid phase chemistry with direct incorporation of phosphothreonine as the N-fluorenylmethoxy-carbonyl-O-benzyl-L-phosphothreonine derivative.

N-terminal or C-terminal fusion proteins comprising a peptide or protein of the invention, conjugated with at least one other molecule, may be prepared by fusing, through recombinant techniques, the N-terminal or C-terminal end of the peptide or protein, and the sequence of a selected protein or selectable marker with a desired biological function. The resultant fusion proteins contain the CSD domain peptide fused to the selected protein or marker protein as described herein. Examples of proteins which may be used to prepare fusion proteins include immunoglobulins and regions thereof, glutathione-S-transferase (GST), hemagglutinin (HA), and truncated myc.

A peptide of the invention may be developed using a biological expression system. The use of these systems allows the production of large libraries of random sequences and the screening of these libraries for sequences that bind to particular peptides. Libraries may be produced by cloning synthetic DNA that encodes random peptide sequences into appropriate expression vectors (see Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

The peptide of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

The present invention further encompasses fusion peptides in which the peptide of the invention or fragments thereof, are recombinantly fused or chemically conjugated (including both covalent and non-covalent conjugations) to heterologous peptides (i.e., an unrelated peptide or portion thereof, e.g., at least 10, at least 20, at least 30, at least 40, at least 50, at least 60, at least 70, at least 80, at least 90, at least 100, at least 125, at least 150, at least 175, at least 200, at least 225, at least 250, at least 275, at least 300, or at least 500 amino acids of the polypeptide) to generate fusion peptides. The fusion does not necessarily need to be direct, but may occur through linker sequences.

In one example, a fusion peptide in which a peptide of the invention or a fragment thereof can be fused to sequences derived from various types of immunoglobulins. For example, a polypeptide of the invention can be fused to a constant region (e.g., hinge, CH2, and CH3 domains) of human IgG or IgM molecule, for example, as described herein, so as to make the fused peptide or fragments thereof more soluble and stable in vivo. In another embodiment, such fusion peptides can be administered to a subject so as to inhibit interactions between a ligand and its receptors in vivo. Such inhibition of the interaction will block or suppress signal transduction which triggers certain cellular responses.

In one aspect, the fusion peptide comprises a polypeptide of the invention which is fused to a heterologous sequence at its N-terminus or C-terminus. In another embodiment, a peptide of the invention can be fused to tag sequences, e.g., a hexa-histidine peptide, such as the tag provided in a pQE vector (QIAGEN, Inc., 9259 Eton Avenue, Chatsworth, Calif., 91311), among others, many of which are commercially available. As described in Gentz, et al., 1989, Proc. Natl. Acad. Sci. USA 86:821-824, for instance, hexa-histidine provides for convenient purification of the fusion protein. Other examples of peptide tags are the hemagglutinin “HA” tag, which corresponds to an epitope derived from the influenza hemagglutinin protein (Wilson, et al., 1984, Cell 37:767) and the “flag” tag (Knappik, et al., 1994, Biotechniques 17(4):754-761). These tags are especially useful for purification of recombinantly produced peptides of the invention.

Methods of introducing and expressing genes into a cell are known in the art. In one embodiment, a peptide can be entrapped in microcapsules prepared by coacervation techniques or by interfacial polymerization, for example, by the use of hydroxymethylcellulose or gelatin-microcapsules, or poly (methylmethacrolate) microcapsules, respectively, or in a colloid system. Colloidal dispersion systems include macromolecule complexes, nano-capsules, microspheres, beads, and lipid-based systems, including oil-in-water emulsions, micelles, mixed micelles, and liposomes.

In one embodiment, the present invention provides an implantable scaffold or device comprising the CSD domain peptide or nucleic acid molecule encoding the CSD domain peptide. For example, in some embodiments, the present invention provides a tissue engineering scaffold, including but not limited to, a hydrogel, electrospun scaffold, polymeric matrix, or the like, comprising the CSD domain peptide or nucleic acid molecule encoding the CSD domain peptide in or on the scaffold.

Nucleic Acid Molecules

In one embodiment, the methods of the invention comprises administering a composition comprising a nucleic acid molecule encoding a CSD domain peptide or subdomain thereof. In one embodiment, the nucleic acid molecule encodes an amino acid sequence of SEQ ID NO:1, SEQ ID NO:2, SEQ ID NO:3, SEQ ID NO:4, SEQ ID NO:5, SEQ ID NO:6, SEQ ID NO:7 or SEQ ID NO:8.

Further, the nucleic acid molecule encodes a peptide having substantial homology to a CSD domain peptide disclosed herein. In some embodiments, the isolated nucleic acid sequence encodes a CSD domain peptide comprising an amino acid sequence having at least about 75%, 80%, 85%, 86%, 87%, 88%, 89%, 90%, 91%, 92%, 93%, 94%, 95%, 96%, 97%, 98%, or 99% sequence homology with an amino acid sequence selected from SEQ ID NO:1, SEQ ID NO:2, SEQ ID NO:3, SEQ ID NO:4, SEQ ID NO:5, SEQ ID NO:6, SEQ ID NO:7 or SEQ ID NO:8.

The isolated nucleic acid may comprise any type of nucleic acid, including, but not limited to DNA, cDNA, and RNA. For example, in one embodiment, the composition comprises an isolated DNA molecule, including for example, an isolated cDNA molecule, encoding a CSD domain peptide. In one embodiment, the composition comprises an isolated RNA molecule encoding a CSD domain peptide.

The nucleic acid molecules of the present invention can be modified to improve stability in serum or in growth medium for cell cultures. Modifications can be added to enhance stability, functionality, and/or specificity and to minimize immunostimulatory properties of the nucleic acid molecule of the invention. For example, in order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect function of the molecule.

Nucleic acids can be produced using various standard cloning and chemical synthesis techniques. Techniques include, but are not limited to nucleic acid amplification, e.g., polymerase chain reaction (PCR), with genomic DNA or cDNA targets using primers (e.g., a degenerate primer mixture) capable of annealing to a CSD encoding sequence. Nucleic acids can also be produced by chemical synthesis (e.g., solid phase phosphoramidite synthesis) or transcription from a gene. The sequences produced can then be translated in vitro, or cloned into a plasmid and propagated and then expressed in a cell (e.g., a host cell such as yeast or bacteria, a eukaryote such as an animal or mammalian cell or in a plant).

Nucleic acids can be included within vectors as cell transfection typically employs a vector. The term “vector,” refers to, e.g., a plasmid, virus, such as a viral vector, or other vehicle known in the art that can be manipulated by insertion or incorporation of a polynucleotide, for genetic manipulation (i.e., “cloning vectors”), or can be used to transcribe or translate the inserted polynucleotide (i.e., “expression vectors”). Such vectors are useful for introducing polynucleotides in operable linkage with a nucleic acid, and expressing the transcribed encoded protein in cells in vitro, ex vivo or in vivo.

A vector generally contains at least an origin of replication for propagation in a cell. Control elements, including expression control elements, present within a vector, are included to facilitate transcription and translation. The term “control element” is intended to include, at a minimum, one or more components whose presence can influence expression, and can include components other than or in addition to promoters or enhancers, for example, leader sequences and fusion partner sequences, internal ribosome binding sites (IRES) elements for the creation of multigene, or polycistronic, messages, splicing signal for introns, maintenance of the correct reading frame of the gene to permit in-frame translation of mRNA, polyadenylation signal to provide proper polyadenylation of the transcript of a gene of interest, stop codons, among others.

Vectors included are those based on viral vectors, such as retroviral (lentivirus for infecting dividing as well as non-dividing cells), foamy viruses (U.S. Pat. Nos. 5,624,820, 5,693,508, 5,665,577, 6,013,516 and 5,674,703; WO92/05266 and WO92/14829), adenovirus (U.S. Pat. Nos. 5,700,470, 5,731,172 and 5,928,944), adeno-associated virus (AAV) (U.S. Pat. No. 5,604,090), herpes simplex virus vectors (U.S. Pat. No. 5,501,979), cytomegalovirus (CMV) based vectors (U.S. Pat. No. 5,561,063), reovirus, rotavirus genomes, simian virus 40 (SV40) or papilloma virus (Cone et al., Proc. Natl. Acad. Sci. USA 81:6349 (1984); Eukaryotic Viral Vectors, Cold Spring Harbor Laboratory, Gluzman ed., 1982; Sarver et al., Mol. Cell. Biol. 1:486 (1981); U.S. Pat. No. 5,719,054). Adenovirus efficiently infects slowly replicating and/or terminally differentiated cells and can be used to target slowly replicating and/or terminally differentiated cells. Simian virus 40 (SV40) and bovine papilloma virus (BPV) have the ability to replicate as extra-chromosomal elements (Eukaryotic Viral Vectors, Cold Spring Harbor Laboratory, Gluzman ed., 1982; Sarver et al., Mol. Cell. Biol. 1:486 (1981)). Additional viral vectors useful for expression include reovirus, parvovirus, Norwalk virus, coronaviruses, paramyxo- and rhabdoviruses, togavirus (e.g., sindbis virus and semliki forest virus) and vesicular stomatitis virus (VSV) for introducing and directing expression of a polynucleotide or transgene in pluripotent stem cells or progeny thereof (e.g., differentiated cells).

Vectors including a nucleic acid can be expressed when the nucleic acid is operably linked to an expression control element. As used herein, the term “operably linked” refers to a physical or a functional relationship between the elements referred to that permit them to operate in their intended fashion. Thus, an expression control element “operably linked” to a nucleic acid means that the control element modulates nucleic acid transcription and as appropriate, translation of the transcript.

The term “expression control element” refers to nucleic acid that influences expression of an operably linked nucleic acid. Promoters and enhancers are particular non-limiting examples of expression control elements. A “promoter sequence” is a DNA regulatory region capable of initiating transcription of a downstream (3′ direction) sequence. The promoter sequence includes nucleotides that facilitate transcription initiation. Enhancers also regulate gene expression, but can function at a distance from the transcription start site of the gene to which it is operably linked. Enhancers function at either 5′ or 3′ ends of the gene, as well as within the gene (e.g., in introns or coding sequences). Additional expression control elements include leader sequences and fusion partner sequences, internal ribosome binding sites (IRES) elements for the creation of multigene, or polycistronic, messages, splicing signal for introns, maintenance of the correct reading frame of the gene to permit in-frame translation of mRNA, polyadenylation signal to provide proper polyadenylation of the transcript of interest, and stop codons.

Expression control elements include “constitutive” elements in which transcription of an operably linked nucleic acid occurs without the presence of a signal or stimuli. For expression in mammalian cells, constitutive promoters of viral or other origins may be used. For example, SV40, or viral long terminal repeats (LTRs) and the like, or inducible promoters derived from the genome of mammalian cells (e.g., metallothionein IIA promoter; heat shock promoter, steroid/thyroid hormone/retinoic acid response elements) or from mammalian viruses (e.g., the adenovirus late promoter; mouse mammary tumor virus LTR) are used.

Expression control elements that confer expression in response to a signal or stimuli, which either increase or decrease expression of operably linked nucleic acid, are “regulatable.” A regulatable element that increases expression of operably linked nucleic acid in response to a signal or stimuli is referred to as an “inducible element.” A regulatable element that decreases expression of the operably linked nucleic acid in response to a signal or stimuli is referred to as a “repressible element” (i.e., the signal decreases expression; when the signal is removed or absent, expression is increased).

Expression control elements include elements active in a particular tissue or cell type, referred to as “tissue-specific expression control elements.” Tissue-specific expression control elements are typically more active in specific cell or tissue types because they are recognized by transcriptional activator proteins, or other transcription regulators active in the specific cell or tissue type, as compared to other cell or tissue types.

The nucleic acid or protein can be stably or transiently transfected (expressed) in the cell and progeny thereof. The cell(s) can be propagated and the introduced nucleic acid transcribed and protein expressed. A progeny of a transfected cell may not be identical to the parent cell, since there may be mutations that occur during replication.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a nucleic acid molecule encoding a peptide or protein into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York).

Biological methods for introducing a nucleic acid molecule encoding a peptide or protein of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a nucleic acid molecule encoding a peptide or protein into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

The vectors of the present invention may also be used for nucleic acid gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

Treatment Regimens

In one embodiment, a composition comprising a CSD domain peptide, or nucleic acid molecule encoding the same, of the invention is administered to a subject. In one embodiment, a treatment regimen may include a single administration of a composition comprising a CSD domain peptide, or nucleic acid molecule encoding the same, of the invention or multiple administrations of a composition comprising a CSD domain peptide, or nucleic acid molecule encoding the same, of the invention. Multiple administrations of at least one composition of the invention can occur sequentially over a period of time selected by the attending physician. Methods of assessment of treatment course are within the skill of the art of an attending physician.

A determination of the need for treatment will typically be assessed by a history and physical exam consistent with the disease or disorder at issue. Subjects with an identified need of therapy include those with diagnosed fibrosis, microvascular leakage, aging and aging-related diseases and disorders, heart disease, and kidney disease. Causes of fibrosis, microvascular leakage, aging and aging-related diseases and disorders, heart disease, and kidney disease include, but are not limited to, genetic disorders, autoimmune disorders, inflammation, damage resulting from injury or trauma, damage from radiation or oxidative free radicals, or exposure to an environmental or medical agent.

In one embodiment, a subject in need of treatment according to the methods described herein will be diagnosed with or be at risk of developing an fibrosis, microvascular leakage, aging and aging-related diseases and disorders, heart disease, and kidney disease. In one embodiment, the subject is an animal, including, but not limited to, mammals (e.g., horses, cows, dogs, cats, sheep, pigs, and humans), reptiles, and avians (e.g., chickens).

It should be recognized that methods of this invention can easily be practiced in conjunction with existing therapies to effectively treat or prevent disease. The methods and compositions of the invention can include concurrent or sequential treatment with non-biologic and/or biologic drugs.

The compositions of the invention may be applied by several routes including systemic administration (e.g., intravenous injection) or by direct administration to the site of intended benefit. Compositions of the invention may be administered using any known administration route, including, but not limited to local, colonic (rectal), topical, nasal and parenteral (including intraperitoneal, subcutaneous, intravenous, intradermal or intramuscular injection, systemically, parenterally, or topically, such as, in oral formulations, inhaled formulations, including solid or aerosol, and by other formulations (including formulations for transdermal, buccal or sublingual administration). In some embodiments, the compositions are formulated for intranasal (i.n.), oro-pharyngeal (o.p.), or intraperitoneal (i.p.) administration.

In one aspect, the invention relates to a method of inhibiting at least one kinase comprising administering a CSD domain peptide of the invention, or nucleic acid molecule encoding a CSD domain peptide of the invention. In one embodiment, the kinase is at least one of TGFβR2, PKCα, PKCε, cMet, VEGFR2, TGFβR1, and Src.

### Pharmaceutical Formulations and Administration

In one aspect, the invention relates to a method of administering a composition comprising a CSD domain peptide of the invention, or nucleic acid molecule encoding a CSD domain peptide of the invention, to a subject who has a disease or disorder described herein. For example, in certain embodiments, the subject has a interstitial lung disease, idiopathic pulmonary fibrosis, lung fibrosis, chronic obstructive pulmonary disease (COPD), Raynaud's phenomenon, pulmonary fibrosis, cirrhosis, atrial fibrosis, endomyocardial fibrosis, arthrofibrosis, Crohn's Disease, mediastinal fibrosis, myelofibrosis, tubulointerstitial fibrosis, hepatic fibrosis, premacular fibrosis, retinal fibrosis, dermal fibrosis, wound-associated fibrosis, Peyronie's disease, nephrogenic systemic fibrosis, progressive massive fibrosis, retroperitoneal fibrosis, fibroma, scleroderma, radiation-induced fibrosis especially due to radiation therapy, atherosclerosis, cardiovascular disease, kidney disease, microvascular leakage, arthritis, cataracts, osteoporosis, hypertension, congestive heart failure, interstitial lung diseases, asthma, kidney failure, neurodegenerative diseases including Alzheimer's disease and vascular dementia, cancer, venous thrombosis, diabetes and complications of diabetes, sepsis, and acute respiratory distress syndrome (ARDS), cardiac hypertrophy, cardiomyopathy, stroke, renal inflammatory injury, chronic kidney failure, and kidney dysfunction.

In one embodiment, the sample is isolated from a subject having or at risk for cancer. In one embodiment, the cancer is breast cancer, however the invention is not limited to detection of a breast cancer. The following are non-limiting examples of cancers that can be diagnosed or treated by the disclosed methods and compositions: acute lymphoblastic leukemia, acute myeloid leukemia, adrenocortical carcinoma, appendix cancer, basal cell carcinoma, bile duct cancer, bladder cancer, bone cancer, brain and spinal cord tumors, brain stem glioma, brain tumor, breast cancer, bronchial tumors, burkitt lymphoma, carcinoid tumor, central nervous system atypical teratoid/rhabdoid tumor, central nervous system embryonal tumors, central nervous system lymphoma, cerebellar astrocytoma, cerebral astrocytoma/malignant glioma, cerebral astrocytotna/malignant glioma, cervical cancer, childhood visual pathway tumor, chordoma, chronic lymphocytic leukemia, chronic myelogenous leukemia, chronic myeloproliferative disorders, colon cancer, colorectal cancer, craniopharyngioma, cutaneous cancer, cutaneous t-cell lymphoma, endometrial cancer, ependymoblastoma, ependymoma, esophageal cancer, ewing family of tumors, extracranial cancer, extragonadal germ cell tumor, extrahepatic bile duct cancer, extrahepatic cancer, eye cancer, fungoides, gallbladder cancer, gastric (stomach) cancer, gastrointestinal cancer, gastrointestinal carcinoid tumor, gastrointestinal stromal tumor (gist), germ cell tumor, gestational cancer, gestational trophoblastic tumor, glioblastoma, glioma, hairy cell leukemia, head and neck cancer, hepatocellular (liver) cancer, histiocytosis, hodgkin lymphoma, hypopharyngeal cancer, hypothalamic and visual pathway glioma, hypothalamic tumor, intraocular (eye) cancer, intraocular melanoma, islet cell tumors, kaposi sarcoma, kidney (renal cell) cancer, langerhans cell cancer, langerhans cell histiocytosis, laryngeal cancer, leukemia, lip and oral cavity cancer, liver cancer, lung cancer, lymphoma, macroglobulinemia, malignant fibrous histiocvtoma of bone and osteosarcoma, medulloblastoma, medulloepithelioma, melanoma, merkel cell carcinoma, mesothelioma, metastatic squamous neck cancer with occult primary, mouth cancer, multiple endocrine neoplasia syndrome, multiple myeloma, mycosis, myelodysplastic syndromes, myelodysplastic/myeloproliferative diseases, myelogenous leukemia, myeloid leukemia, myeloma, myeloproliferative disorders, nasal cavity and paranasal sinus cancer, nasopharyngeal cancer, neuroblastoma, non-hodgkin lymphoma, non-small cell lung cancer, oral cancer, oral cavity cancer, oropharyngeal cancer, osteosarcoma and malignant fibrous histiocytoma, osteosarcoma and malignant fibrous histiocytoma of bone, ovarian, ovarian cancer, ovarian epithelial cancer, ovarian germ cell tumor, ovarian low malignant potential tumor, pancreatic cancer, papillomatosis, paraganglioma, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm, plasma cell neoplasm/multiple myeloma, pleuropulmonary blastoma, primary central nervous system cancer, primary central nervous system lymphoma, prostate cancer, rectal cancer, renal cell (kidney) cancer, renal pelvis and ureter cancer, respiratory tract carcinoma involving the nut gene on chromosome 15, retinoblastoma, rhabdomyosarcoma, salivary gland cancer, sarcoma, sezary syndrome, skin cancer (melanoma), skin cancer (nonmelanoma), skin carcinoma, small cell lung cancer, small intestine cancer, soft tissue cancer, soft tissue sarcoma, squamous cell carcinoma, squamous neck cancer, stomach (gastric) cancer, supratentorial primitive neuroectodermal tumors, supratentorial primitive neuroectodermal tumors and pineoblastoma, T-cell lymphoma, testicular cancer, throat cancer, thymoma and thymic carcinoma, thyroid cancer, transitional cell cancer, transitional cell cancer of the renal pelvis and ureter, trophoblastic tumor, urethral cancer, uterine cancer, uterine sarcoma, vaginal cancer, visual pathway and hypothalamic glioma, vulvar cancer, waldenstrom macroglobulinemia, and wilms tumor.

Administration of one or more compositions of the present invention to a subject may be carried out using known procedures, at dosages and for periods of time effective to prevent or treat diseases or disorders involving microvascular leakage, kidney disease, heart disease, and age-related diseases or disorders in the subject. An effective amount of the one or more therapeutic compositions necessary to achieve a therapeutic effect may vary according to factors such as the state of the disease or disorder in the subject, and the age, sex, and weight of the subject. The regimen of administration may affect what constitutes an effective amount.

The dosages of the one or more compositions may be proportionally increased or decreased as indicated by the exigencies of the therapeutic situation. One of ordinary skill in the art would be able to study the relevant factors and make the determination regarding the effective amount of the therapeutic compound without undue experimentation.

Actual dosage levels of the active ingredients in the one or more pharmaceutical compositions of this invention may be varied so as to obtain an amount of the active ingredient that is effective to achieve the desired therapeutic response for a particular subject, composition, and mode of administration, without being toxic to the subject.

In particular, the selected dosage level will depend upon a variety of factors including the activity of the particular one or more compositions employed, the time of administration, the rate of excretion of the one or more compositions, the duration of the treatment, other drugs, compounds or materials used in combination with the one or more compositions, the age, sex, weight, condition, general health and prior medical history of the subject being treated, and like factors well known in the medical arts.

A medical doctor, e.g., physician or veterinarian, having ordinary skill in the art may readily determine and prescribe the effective amount of the one or more pharmaceutical compositions required. For example, the physician or veterinarian could start doses of the one or more compounds of the invention employed in the pharmaceutical composition at levels lower than that required in order to achieve the desired therapeutic effect and gradually increase the dosage until the desired effect is achieved.

Typically, dosages which may be administered in a method of the invention to a subject range in amount from 0.5 ng to about 50 mg per kilogram of body weight of the subject, while the precise dosage administered will vary depending upon any number of factors, including but not limited to, the type of subject and type of disease state being treated, the age of the subject and the route of administration. In one embodiment, the dosage of the compound will vary from about 1 ng to about 10 mg per kilogram of body weight of the subject. In one embodiment, the dosage will vary from about 3 ng to about 1 mg per kilogram of body weight of the subject.

To improve bioavailability and reduce the complications associated with repeated injections, the present invention contemplates sustained delivery of the compositions of the invention alone or in combination with other medications. The sustained delivery in the present invention can be achieved through a number of different delivery systems, including but not limited to polymeric gels, colloidal systems including liposomes and nanoparticles, cyclodextrins, collagen shields, diffusion chambers, flexible carrier strips, and implants.

The compositions of the present invention can be in the form of ointments. Ointments have the benefit of providing prolonged drug contact time with a surface. Ointments will generally include a base comprised of, for example, white petrolatum and mineral oil, often with anhydrous lanolin, polyethylene-mineral oil gel, and other substances recognized by the formulation chemist as being non-irritating, which permit diffusion of the drug, and which retain activity of the medicament for a reasonable period of time under storage conditions.

Therapeutic amounts of a composition of the invention can be administered orally. For these oral dosage forms, the composition may be formulated with a pharmaceutically acceptable solid or liquid carrier. Solid form preparations include powders, tablets, pills, capsules, cachets, and dispersible granules. The concentration or effective amount of the composition to be administered per dosage is widely dependent on the actual composition. However, a total oral daily dosage normally ranges from about 50 mg to 30 g, and in certain embodiments from about 250 mg to 25 g. A solid carrier can be one or more substances which may also function as a diluent, a flavoring agent, a solubilizer, a lubricant, a suspending agent, a binder, a preservative, a tablet disintegrating aid, or an encapsulating material. Suitable carriers include magnesium carbonate, magnesium stearate, talc, sugar, lactose, pectin, dextrin, starch gelatin, tragacanth, methylcellulose, sodium carboxymethylcellulose, microcrystalline cellulose, a low melting wax, cocoa butter, and the like. The term “preparation” is intended to include the formulation of the active compound with encapsulating material as a carrier providing a capsule in which the active component, with or without other carriers, is surrounded by a carrier, which is thus in association with it. Similarly, cachets and lozenges are included. Tablets, powders, capsules, pills cachets, and lozenges can be used as solid dosage forms suitable for oral administration.

For administration by inhalation, the compounds according to the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example, a powder mix of the compound and a suitable powder base such as lactose or starch. The powder composition may be presented in unit dosage form in, for example, capsules or cartridges or, e.g., gelatin or blister packs from which the powder may be administered with the aid of an inhalator or insufflator.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

Formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 nanometers to about 2000 micrometers, and may further comprise one or more of the additional ingredients described herein.

A composition may also be contained within an inert matrix for either direct application or injection into the subject. As one example of an inert matrix, liposomes may be prepared from dipalmitoyl phosphatidylcholine (DPPC), for example, prepared from egg phosphatidylcholine (PC) since this lipid has a low heat transition. Liposomes are made using standard procedures as known to one skilled in the art. A composition, in amounts ranging from nanogram to microgram quantities, is added to a solution of egg PC, and the lipophilic drug binds to the liposome.

A time-release drug delivery system may be employed to result in sustained release of the active agent (e.g., a CSD domain peptide or subdomain thereof) over a period of time. The time-release formation may be in the form of a capsule of a polymer (e.g., polycaprolactone, poly(glycolic) acid, poly(lactic) acid, polyanhydride) or lipids that may be formulated as microspheres. The composition bound with liposomes may be applied directly, either in the form of drops or as an aqueous based cream, or may be injected. In a formulation for direct application, the drug is slowly released over time as the liposome capsule degrades due to wear and tear. In a formulation for injection, the liposome capsule degrades due to cellular digestion. Both of these formulations provide advantages of a slow release drug delivery system, allowing the subject a constant exposure to the drug over time.

In a time-release formulation, the microsphere, capsule, liposome, etc. may contain a concentration of a composition that could be toxic if administered as a bolus dose. The time-release administration, however, is formulated so that the concentration released at any period of time does not exceed a toxic amount. This is accomplished, for example, through various formulations of the vehicle (coated or uncoated microsphere, coated or uncoated capsule, lipid or polymer components, unilamellar or multilamellar structure, and combinations of the above, etc.). Other variables may include the subject's pharmacokinetic-pharmacodynamic parameters (e.g., body mass, gender, plasma clearance rate, hepatic function, etc.). The formation and loading of microspheres, microcapsules, liposomes, etc. and their implantation are standard techniques known by one skilled in the art.

Multiple compositions of the invention may be administered simultaneously, separately or spaced out over a period of time so as to obtain the maximum efficacy of the combination; it being possible for each administration to vary in its duration from a rapid administration to a continuous perfusion. As a result, for the purposes of the present invention, the combinations are not exclusively limited to those which are obtained by physical association of the constituents, but also to those which permit a separate administration, which can be simultaneous or spaced out over a period of time.

The administration of a nucleic acid encoding a CSD domain peptide or subdomain thereof of the invention to the subject may be accomplished using gene therapy. Gene therapy is based on inserting a therapeutic gene into a cell by means of an ex vivo or an in vivo technique. Suitable vectors and methods have been described for genetic therapy in vitro or in vivo, and are known as expert on the matter; see, for example, Giordano, Nature Medicine 2 (1996), 534-539; Schaper, Circ. Res 79 (1996), 911-919; Anderson, Science 256 (1992), 808-813; Isner, Lancet 348 (1996), 370-374; Muhlhauser, Circ. Res 77 (1995), 1077-1086; Wang, Nature Medicine 2 (1996), 714-716; WO94/29469; WO97/00957 or Schaper, Current Opinion in Biotechnology 7 (1996), 635-640 and the references quoted therein. A polynucleotide, or a polynucleotide encoding a peptide of the invention, can be designed for direct insertion or by insertion through liposomes or viral vectors (for example, adenoviral or retroviral vectors) into the cell. Suitable gene distribution systems that can be used according to the invention may include liposomes, distribution systems mediated by receptor, naked DNA and viral vectors such as the herpes virus, the retrovirus, the adenovirus and adeno-associated viruses, among others. The distribution of nucleic acids to a specific site in the body for genetic therapy can also be achieved by using a biolistic distribution system, such as that described by Williams (Proc. Natl. Acad. Sci. USA, 88 (1991), 2726-2729). The standard methods for transfecting cells with recombining DNA are well known by an expert on the subject of molecular biology, see, for example, WO94/29469. Genetic therapy can be carried out by directly administering the recombining DNA molecule or the vector of the invention to a patient.

### Cancer Therapeutic

In one embodiment, the invention provides a method to treat cancer metastasis comprising administering a CSD domain peptide of the invention to a subject in need thereof. In some embodiments, the CSD domain peptide cam be administered in combination with a complementary therapy for the cancer, such as surgery, chemotherapy, chemotherapeutic agent, radiation therapy, or hormonal therapy or a combination thereof.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

The CSD domain peptides of the invention can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the present disclosure include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the disclosed compounds include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out exemplary embodiments of the present invention, and are not to be construed as limiting in any way the remainder of the disclosure.

### Example 1: CSD Domains in Treating Angiotensin II-Induced Heart Disease

The data presented herein demonstrates that CSD domain peptides (Table 1) are very active in suppressing heart disease and microvascular leakage in mice in which these pathologies had been induced by treatment with angiotensin II.

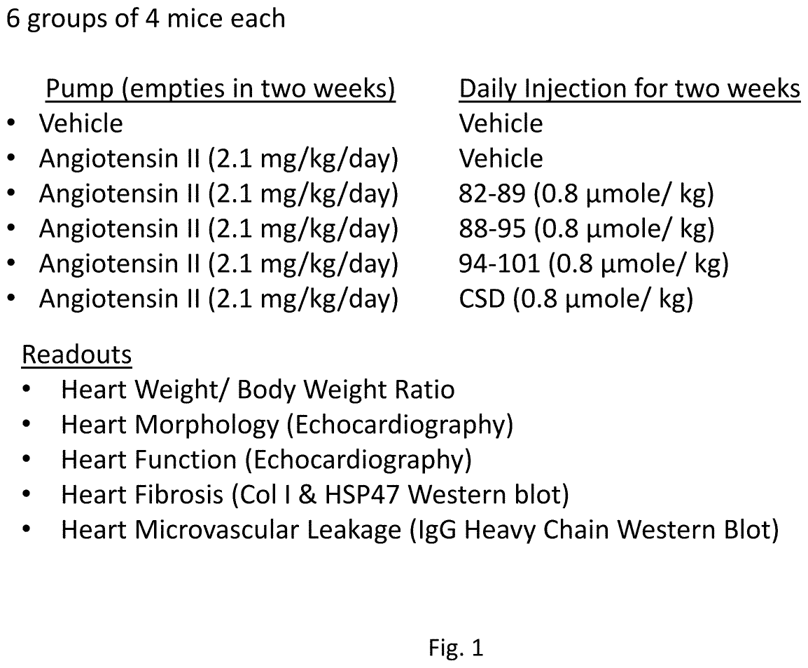

FIG. 1 provides an overview of an experiment using Full-Length CSD, 82-89, 88-95, and 94-101 (SEQ ID NO:1, SEQ ID NO:2, SEQ ID NO:3, and SEQ ID NO:4 respectively).

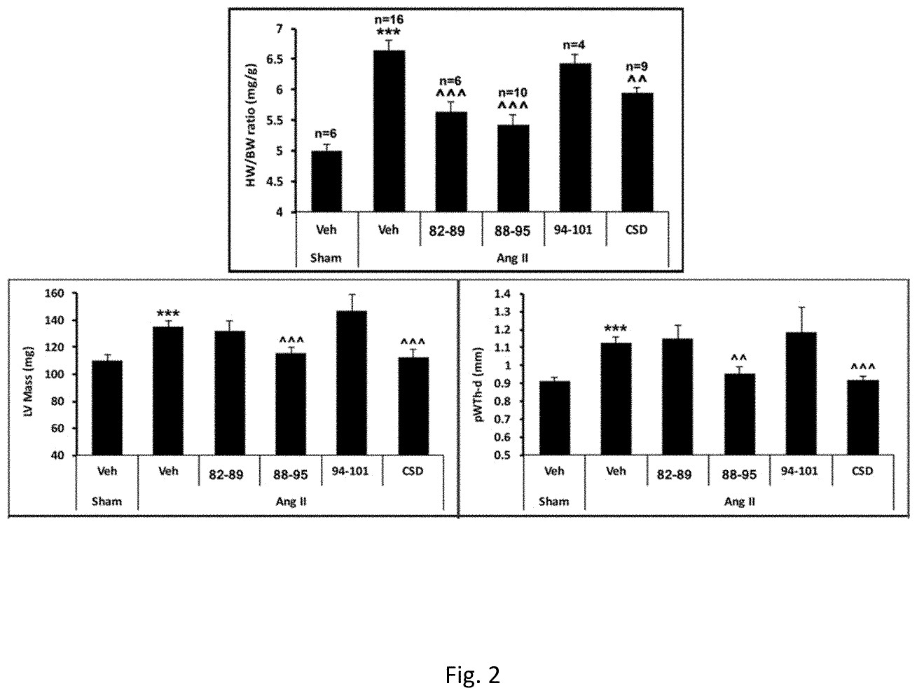

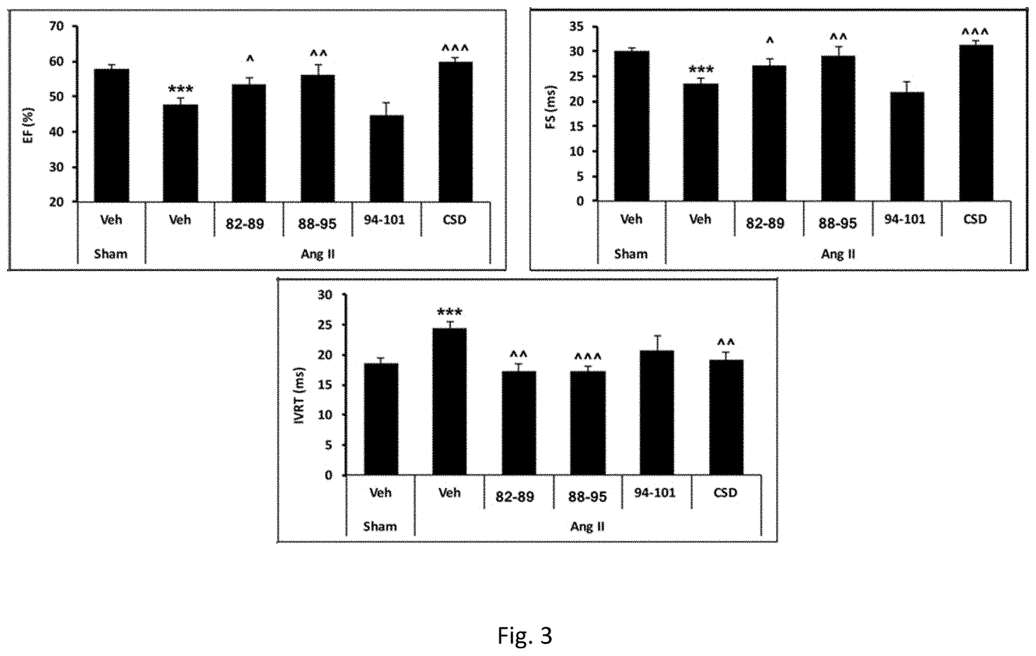

Highly significant changes in Heart Weight/Body Weight (HW/BW), Left Ventricle (LV) Mass, and pWTh (Posterior Wall Thickness) are induced by Ang II (FIG. 2). AngII also induces pathological changes in Ejection Fraction (EF), Fractional Shortening (FS), and Isovolumic Relaxation Time (IVRT) (FIG. 3). For each of these parameters, 88-95 is the most effective CSD subdomain in suppressing the effects of Ang II. However, 82-89 is essentially equally effective as 88-95 in suppressing the effects of AngII on HW/BW and has a significant beneficial effect on EF, FS, and IVRT.

FIG. 2 and FIG. 3 provide data demonstrating that CSD and two subdomains suppress Ang II-induced increased HW/BW ratio and pathological changes in ventricular function. Young mice (3 months old) infused with Ang II or vehicle for two weeks received daily i.p. injections of CSD, the indicated subdomains or scrambled CSD (0.8 μmol/kg). Mice were evaluated by Echocardiography for changes in posterior wall thickness (pWTh), fractional shortening (FS), cardiac output (CO), ejection fraction (EF), and isovolumic relaxation time (IVRT). Significant changes are shown as ***p<0.001 for Sham+Veh vs AngII+Veh. Significant suppression of AngII-induced changes is shown as \hat{ }p<0.05, \hat{ }\hat{ }p<0.01, and \hat{ }\hat{ }\hat{ }p<0.001 for AngII+Veh vs AngII+CSD or AngII+CSD subdomains. The number of mice used in each treatment are shown in the HW/BW data.

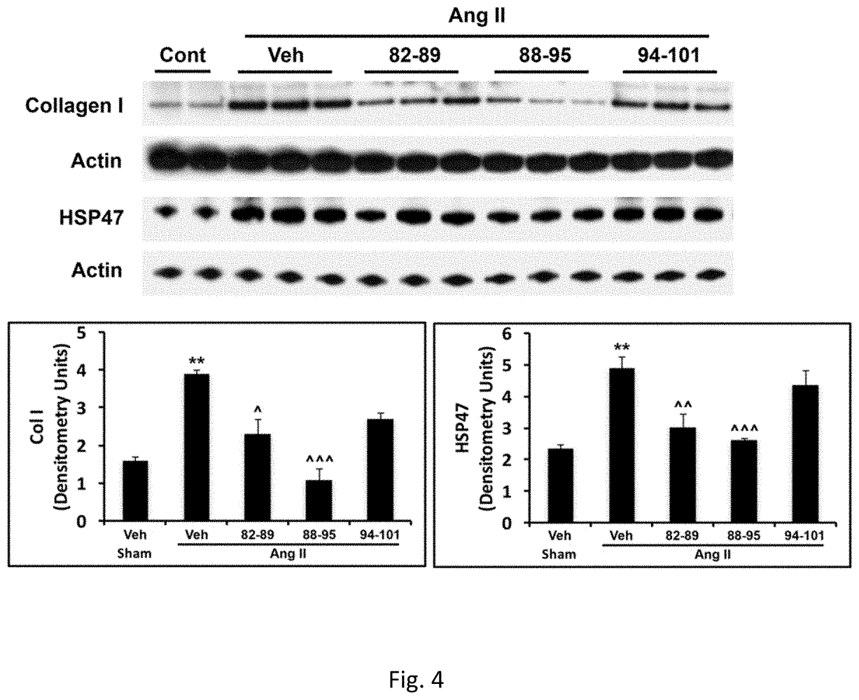

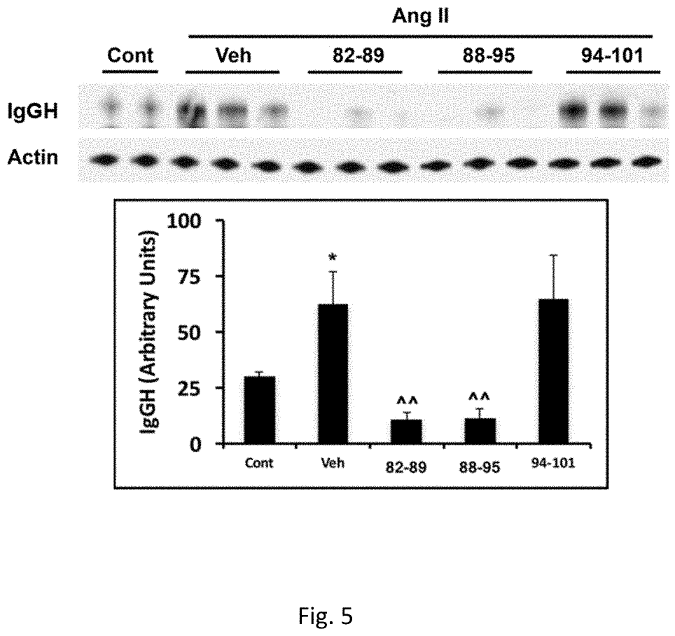

In the heart, AngII induces fibrosis measured in terms of increased Col I deposition and HSP47 level (FIG. 4). For both parameters, 88-95 is the most effective CSD subdomain in suppressing the effects of AngII although 82-89 also has a significant beneficial effect. AngII induces microvascular leakage in the heart (measured in terms of IgG heavy chain level in the tissue) that is almost completely suppressed both by 82-89 and 88-95 (FIG. 5).

FIGS. 4 and 5 show that two CSD subdomains significantly suppress AngII-Induced microvascular leakage and fibrosis in the heart. Young mice (3 months old) infused with Ang II or vehicle for two weeks received daily i.p. injections of the indicated subdomains of CSD. The results of a typical experiment for Col I and HSP47 (FIG. 4) and for microvascular leakage (FIG. 5) are shown in Western blots (2 to 3 mice per group). The data are quantified below (n=4). Significant changes are shown as *p<0.05 and **p<0.01 for Sham+Veh vs Ang II+Veh. Significant suppression of Ang II-induced changes by subdomains are shown as \hat{ }p<0.05, \hat{ }\hat{ }V 0.01, and \hat{ }\hat{ }\hat{ }p<0.001 for Ang II+Veh vs Ang II+CSD subdomains.

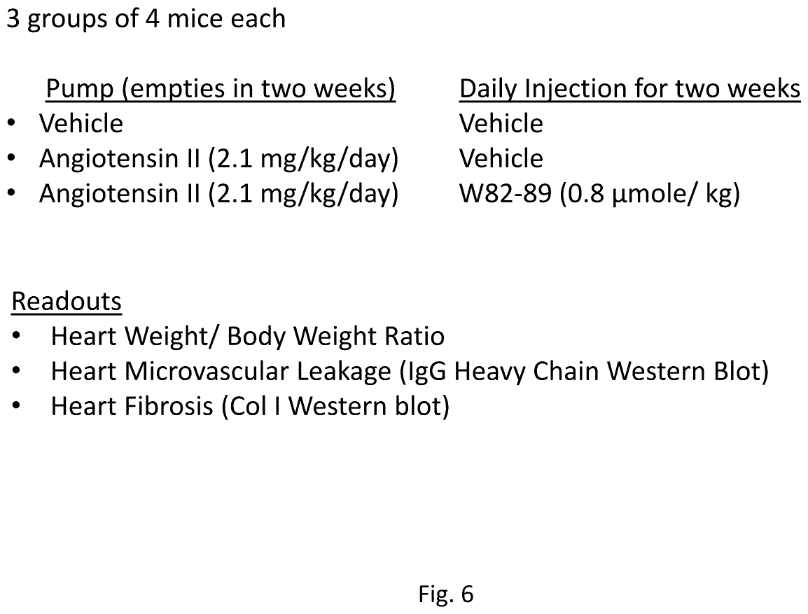

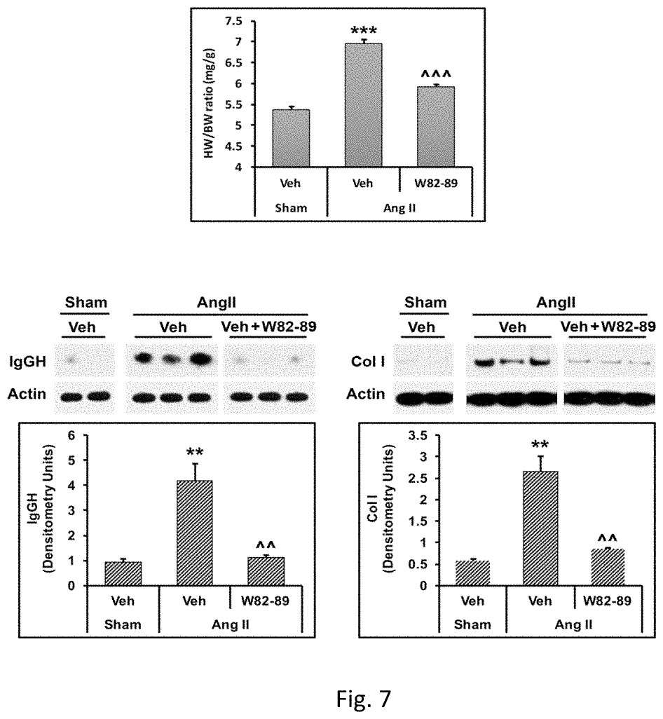

FIG. 6 provides an overview of an experiment using W82-89 (SEQ ID NO: 6). As above, AngII induces highly significant changes in HW/BW ratio, microvascular leakage, and fibrosis (FIG. 7). All of these AngII-induced pathologies are suppressed by W82-89 with high significance.

FIG. 7 shows that W82-89 suppresses AngII-induced pathological increases in HW/BW ratio, microvascular leakage, and Col I levels in the heart. Young mice (3 months old) infused with Ang II or vehicle for two weeks received daily i.p. injections of W82-89. IgG heavy chain (IgGH) leakage and Col I accumulation are shown in Western blots. The actin loading control is present twice because IgGH was analyzed in the supernatant fraction of heart homogenate while Col I is found in the pellet fraction. In the quantitation (n=4), significant changes are shown as **p<0.01 and ***p<0.001 for Sham+Veh vs Ang II+Veh and \hat{ }\hat{ }p<0.01 and \hat{ }\hat{ }\hat{ }p<0.001 for reversal of AngII-induced changes by W82-89.

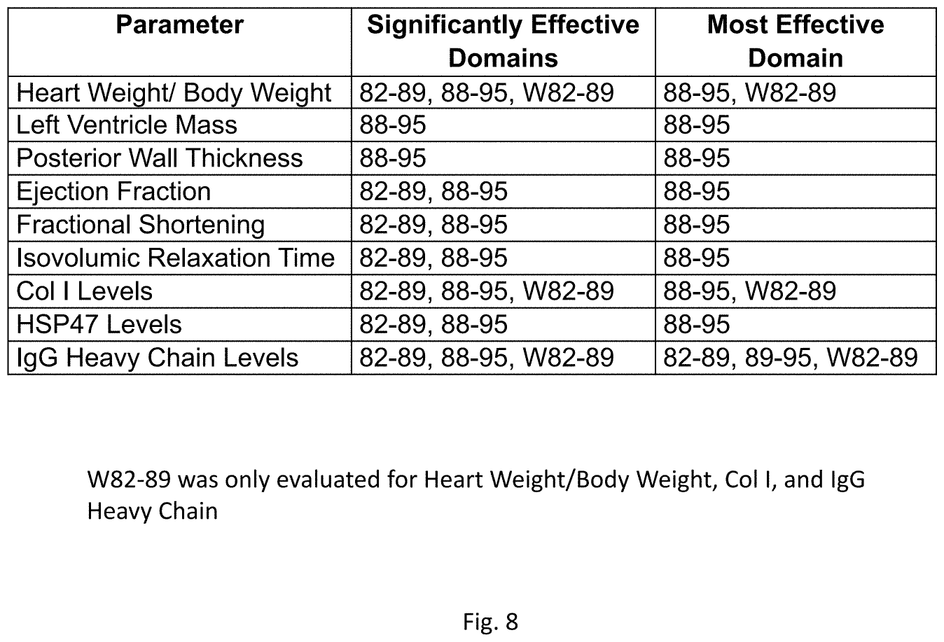

FIG. 8 provides a summary of the data from FIGS. 1 through 7. 88-95 is the most effective Dimethyl sulfoxide (DMSO)-solubilized domain tested. However, modification of 82-89 to make it water soluble (W82-89) makes it more effective than 82-89 and at least as effective as 88-95. Thus, both 82-89 and 88-95 are active domains within CSD.

TABLE 1
CSD domain peptides

| CSD domain peptide | Sequence | SEQ ID NO |
|---|---|---|
| Full-Length CSD | DGIWKASFTTFTVTKY | SEQ ID NO: 1 |
| (caveolin-1 aa82-101) | WFYR-NH2 | |
| Caveolin-1 aa82-89 | DGIWKASF-NH2 | SEQ ID NO: 2 |
| Caveolin-1 aa88-95 | SFTTFTVT-NH2 | SEQ ID NO: 3 |
| Caveolin-1 aa94-101 | VTKYWFYR-NH2 | SEQ ID NO: 4 |
| Modified Full-Length | Ac-kkDGIWKASFTTF | SEQ ID NO: 5 |
| CSD | TVTKYWFYRkk-NH2 | |
| Modified aa82-89 | Ac-kkDGIWKASFkk- | SEQ ID NO: 6 |
| | NH2 | |
| Modified aa88-95 | Ac-kkSFTTFTVTkk- | SEQ ID NO: 7 |
| | NH2 | |
| Modified aa94-101 | Ac-kkVTKYWFYRkk- | SEQ ID NO: 8 |
| | NH2 | |

Lower-case letters indicate D-amino acids that are not part of caveolin-1. For injection, SEQ ID NO: 1, SEQ ID NO: 2, SEQ ID NO: 3, and SEQ ID NO: 4 are dissolved in very small volumes of dimethyl sulfoxide (DMSO) then diluted 100-fold with saline. SEQ ID NO: 5, SEQ ID NO: 6, SEQ ID NO: 7, and SEQ ID NO: 8 are water-soluble so are dissolved directly in saline. Because of their solubility in water, they are sometimes referred to as WCSD (SEQ ID NO: 5), W82-89 (SEQ ID NO: 6), W88-95 (SEQ ID NO: 7), and W94-101 (SEQ ID NO: 8) respectively.

### Example 2: CSD Reverses Aging-Associated Pathological Changes in the Heart and Kidney

The data presented herein demonstrates that CSD peptides (Table 1) are very active in suppressing heart and kidney disease and microvascular leakage in mice in which these pathologies have occurred due to aging.

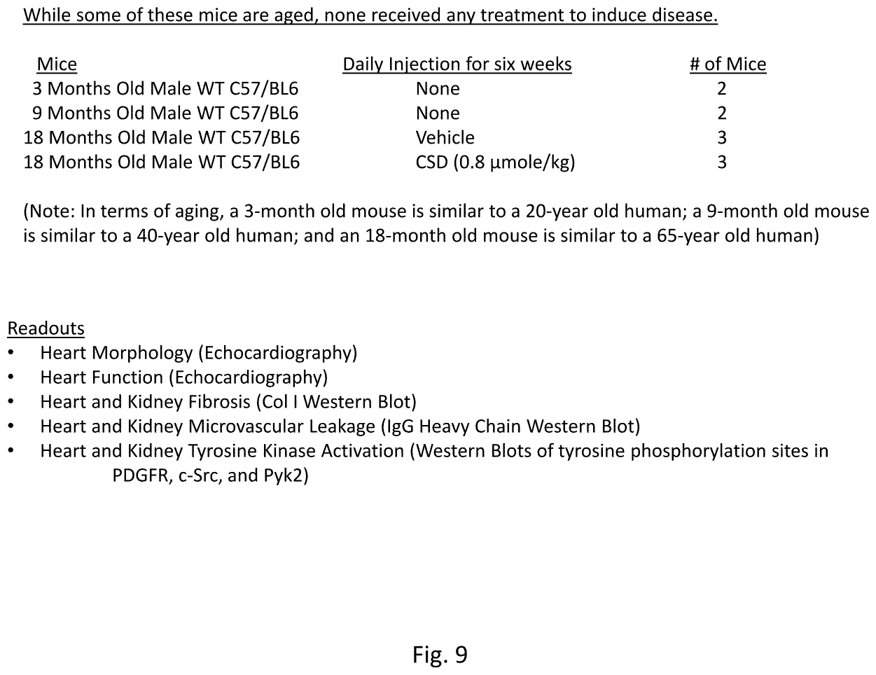

FIG. 9 provides an overview of an experiment using CSD (SEQ ID NO: 1).

The function of various organs in the elderly goes downhill progressively. This is in part due to increases in fibrosis and microvascular leakage. The experiments presented in this Example have examined these progressive processes in mice. The results demonstrate that when 18-month old mice (an age similar to a 65-year old human) are treated daily with CSD for six weeks, the levels of fibrosis and microvascular leakage in the heart and kidney decrease to the levels observed in healthy 3-month old mice (an age similar to a 20-year old human).

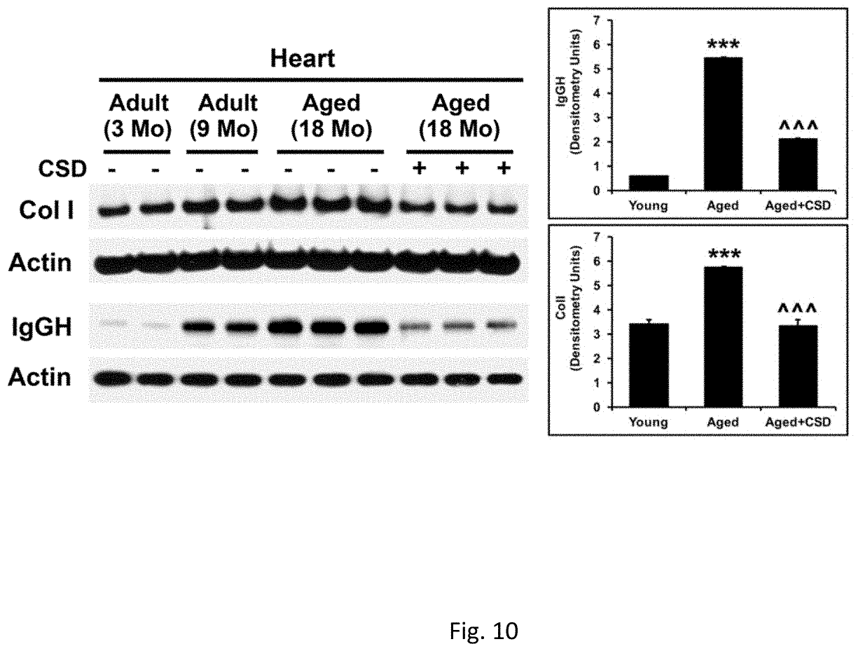

Both fibrosis and microvascular leakage in the heart progress with aging from 3 months to 9 months to 18 months. CSD treatment of 18-month old mice reverses the levels of fibrosis and microvascular leakage almost to the level observed in 3-month old mice (FIG. 10).

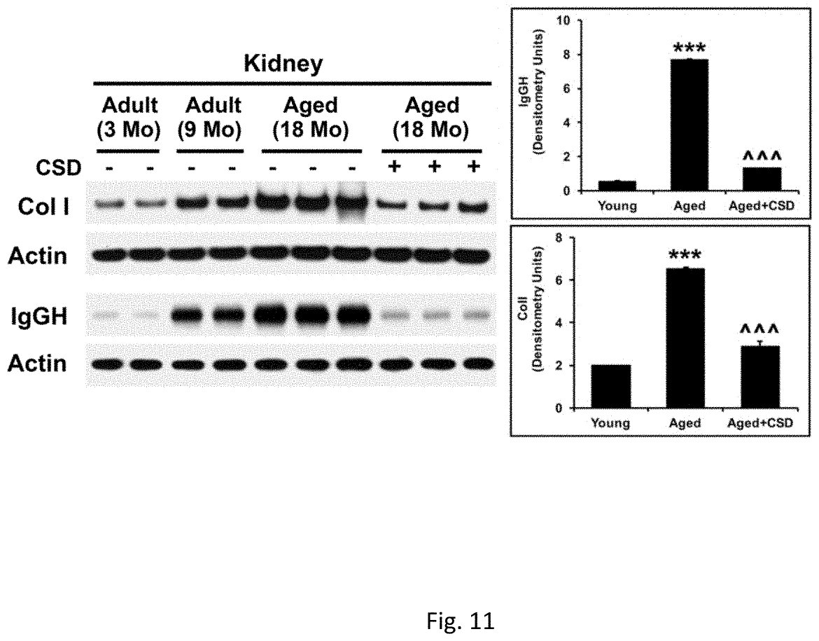

Both fibrosis and microvascular leakage in the kidney progress with aging from 3 months to 9 months to 18 months. CSD treatment of 18-month old mice reverses the levels of fibrosis and microvascular leakage almost to the level observed in 3-month old mice (FIG. 11).

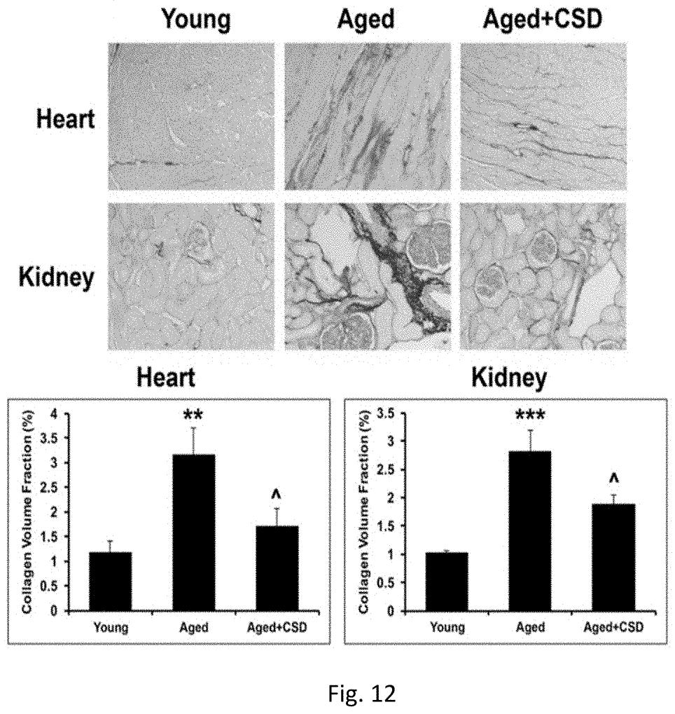

To further validate the reduced fibrosis in CSD-treated aged mice, heart and kidney tissue sections were stained with picrosirius red and the collagen volume fraction determined (FIG. 12). Compared to young mice, aged mice showed a high level of picrosirius red staining in both the heart and kidney that was significantly reduced by CSD treatment.

FIG. 10, FIG. 11, and FIG. 12 demonstrate that microvascular leakage and fibrosis associated with aging is reversed by CSD in the heart and kidney.

Young C57/Bl6 mice (3 months), middle-aged C57/Bl6 mice (9 months), and aged C57/Bl6 mice (18 months) were injected daily i.p. for six weeks with saline vehicle or CSD. IgG heavy chain leakage and Col I accumulation were evaluated by Western blots of heart (FIG. 10) and kidney tissue (FIG. 11). Actin was the loading control. Two to three mice per category are shown. In the quantifications (n=4), significant changes are shown as ***p<0.001 between young and aged mice and \hat{ }\hat{ }\hat{ }p<0.001 for decreases in aged mice treated with CSD. In FIG. 12, representative examples of picrosirius red staining of heart and kidney tissue sections from the indicated mice are shown. The data were quantified (n=3) as in terms of Collagen Volume Fraction. Significant changes are shown as ***p<0.001 and **p<0.01 between young and aged mice and \hat{ }p<0.05 for decreases in aged mice treated with CSD.

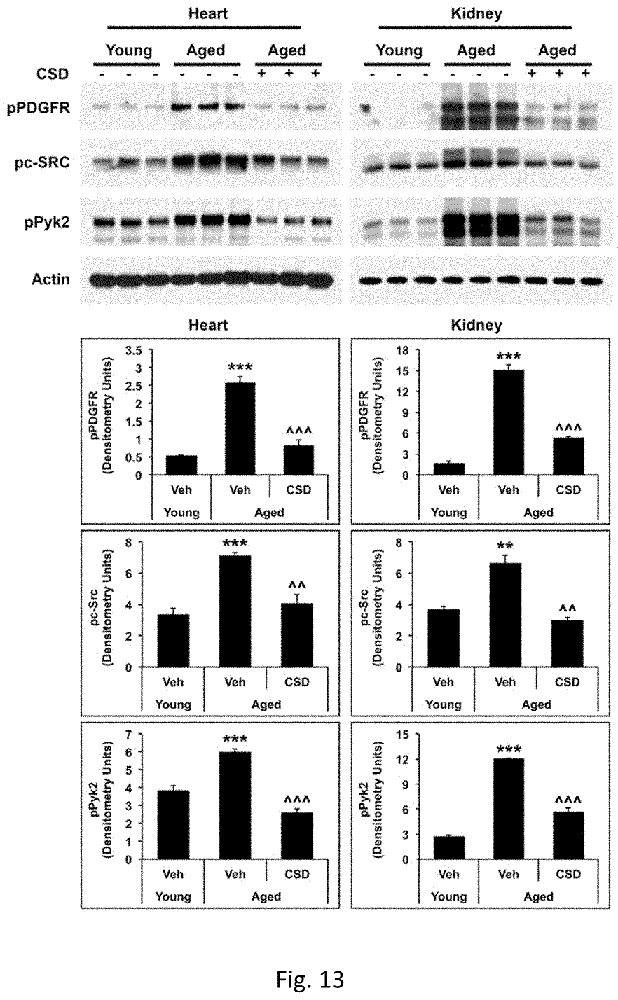

Because tyrosine kinase activation (phosphorylation) has been implicated in vascular hyperpermeability due to its effect on junctional proteins, the effect of CSD on the activation of receptor and non-receptor tyrosine kinases was examined (FIG. 13). Similar to the changes that occurred in microvascular leakage and fibrosis, both in heart and kidney a major aging-associated increase in the activation of the receptor tyrosine kinase PDGFR and the non-receptor tyrosine kinases c-Src and Pyk2 was observed. Again, these aging-associated changes were almost completely reversed by CSD.

FIG. 13 demonstrates that tyrosine kinase activation in aged mouse heart and kidney is reversed by CSD. The same extracts used in FIG. 10 and FIG. 11 were analyzed by Western Blot for tyrosine kinase activation using antibodies specific for phosphorylated tyrosine residues in PDGFR (α-Y849/β-Y857), c-Src Y426, and Pyk2 Y402. Actin was the loading control. In the quantifications, significant changes are shown as ***p<0.001 and **p<0.01 between young and aged mice and \hat{ }\hat{ }\hat{ }p<0.001 and \hat{ }\hat{ }p<0.01 for decreases in Aged mice treated with CSD.

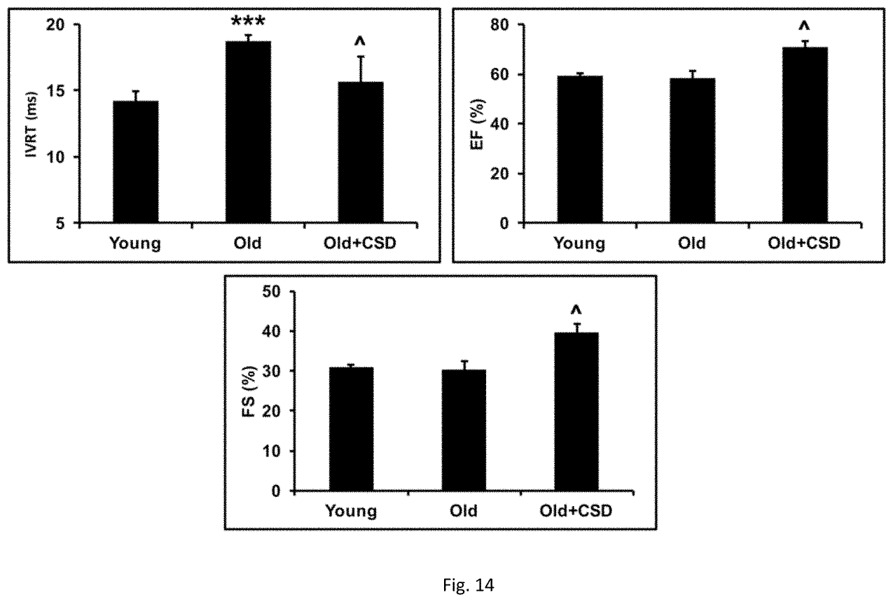

Given the beneficial effects of CSD on microvascular leakage, fibrosis, and tyrosine kinase signaling in the heart, the effects of CSD on ventricular function were evaluated by Echocardiography. For FS and SV, while no significant difference was observed between young and aged mice, CSD had a positive effect on these parameters (FIG. 14). For IVRT (a measurement of diastolic function), aging caused a prolonged ventricular relaxation time that was resolved by CSD treatment (FIG. 14). These observations suggest that cardiac function in these mice is similar to cardiac function in human patients with “Heart Failure with Preserved Ejection Fraction (diastolic heart failure or HFpEF)”, the most common form of heart failure in the elderly.

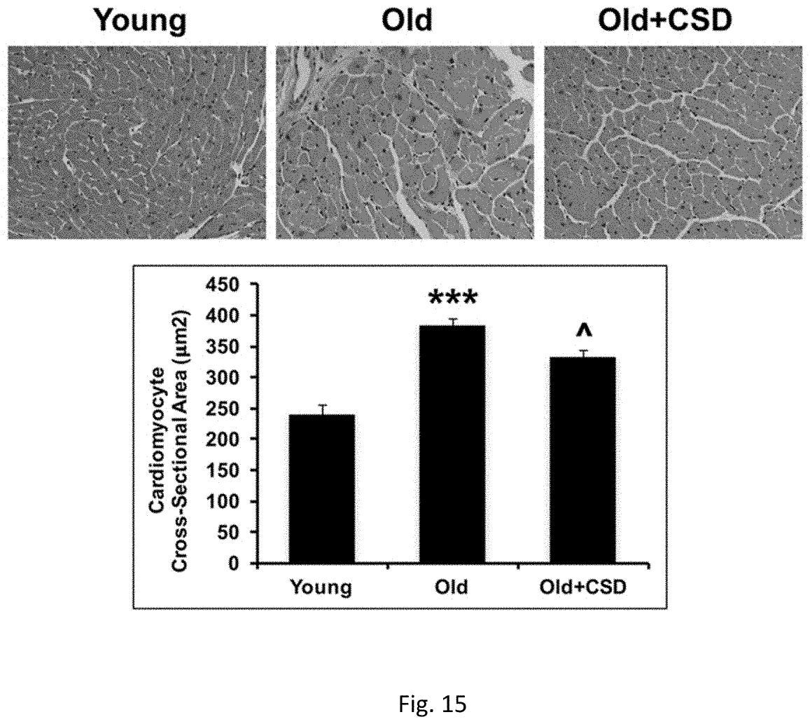

Because young mice treated with AngII undergo cardiomyocyte hypertrophy that is reversed by CSD, we also evaluated this parameter in aged mice. Indeed, as in AngII-treated mice, cardiomyocyte hypertrophy was increased in aged mice and this increase was reversed by CSD (FIG. 15).

CSD Improves Ventricular Function in Aged Mice.

Echocardiographic analyses were performed on the same mice used in FIG. 10 and FIG. 11 prior to their sacrifice. M-mode echo measurements in parasternal short-axis (PSAX) view were used to quantify changes in EF and FS. Tissue Doppler measurements in PSAX were used to measure IVRT. Values are shown as Mean±SEM. Statistically significant changes are shown as ***p<0.001 between young and aged mice and \hat{ }p<0.05 for improved functions in aged mice due to CSD treatment (FIG. 14).

CSD Reverses Cardiac Hypertrophy Associated with Aging.

Left ventricle tissue sections from the same mice used in FIG. 10 were stained with hematoxylin-eosin and cardiomyocyte cross-sectional area was quantified by measuring at least 50 cardiomyocytes for each group (n=3) using SigmaScan Pro image analysis. Statistical significance is shown as ***p<0.001 between young and aged mice and \hat{ }p<0.05 for decreased hypertrophy in aged mice due to CSD treatment (FIG. 15).

### Example 3: CSD Reverses Aging-Associated Pathological Changes in the Brain

The data presented herein demonstrates that CSD peptides (Table 1) are very active in suppressing brain disease and microvascular leakage in mice in which these pathologies have occurred due to aging.

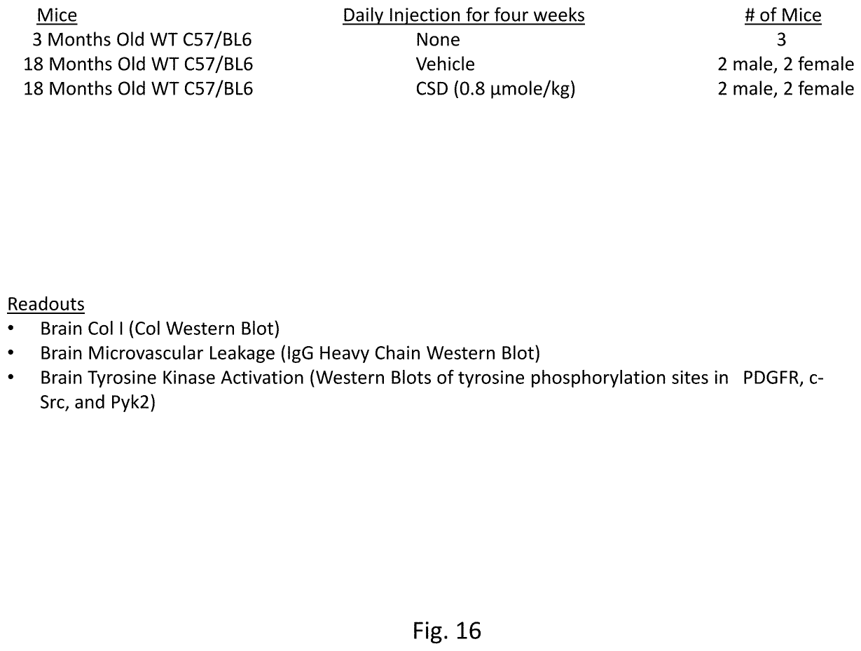

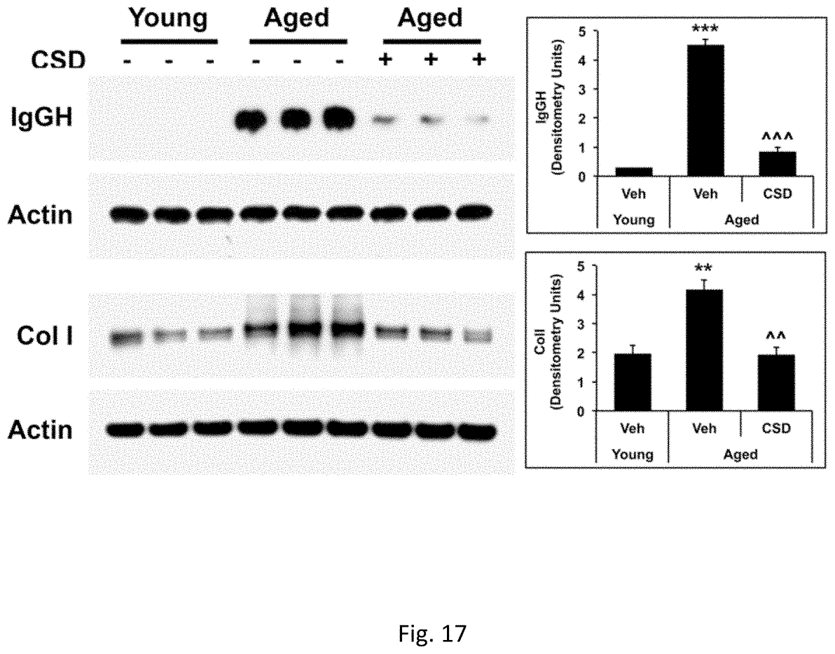

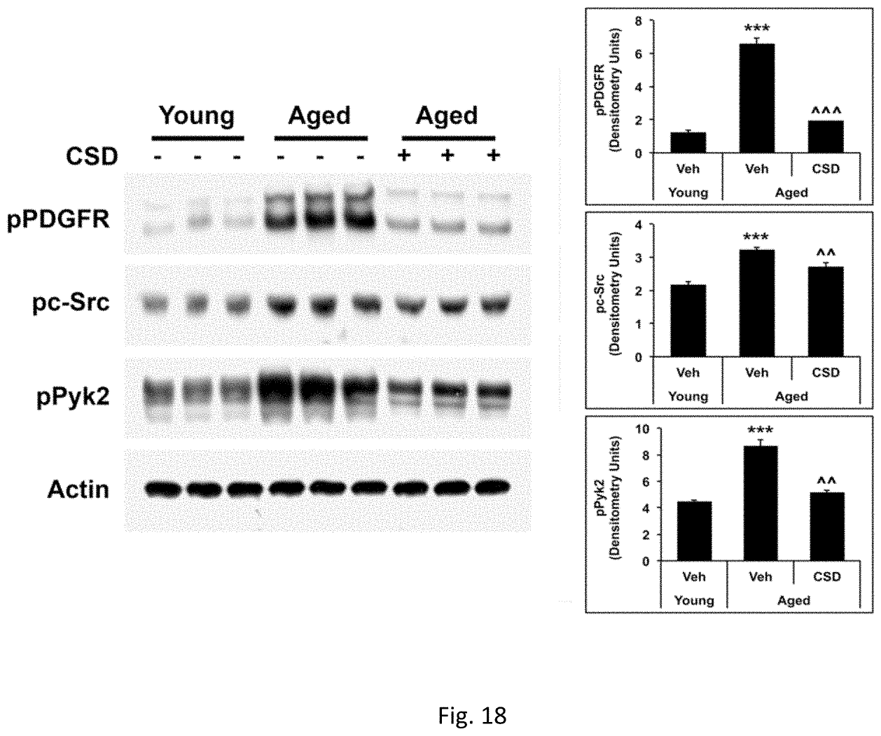

FIG. 16 provides an overview of an experiment using CSD (SEQ ID NO: 1). It is similar to the experiment described in FIG. 9 except that the readouts were performed on brain tissue rather than heart and kidney tissue. The results demonstrate that 18-month old mice have much higher levels in the brain than young mice of microvascular leakage and fibrosis (FIG. 17), and of activated tyrosine kinases (FIG. 18) and that systemic CSD treatment decreases these levels almost to the levels observed in healthy 3-month old mice.

Microvascular Leakage, Fibrosis, and Tyrosine Kinase Activation Associated with Aging in the Brain are Reversal by CSD.

Mice were treated as described in FIG. 10, except that CSD injections were for four weeks. Brain tissue extracts were analyzed by Western blot for microvascular leakage and fibrosis (FIG. 17) and for tyrosine kinase activation (FIG. 18). In the quantifications (n=3), significant changes are shown as ***p<0.001 and **p<0.01 between young and aged mice and \hat{ }\hat{ }\hat{ }p<0.001 and \hat{ }\hat{ }p<0.01 for decreases in aged mice due to CSD treatment.

### Example 4: Suppression of Lung and Skin Fibrosis by Unmodifed CSD Subdomains

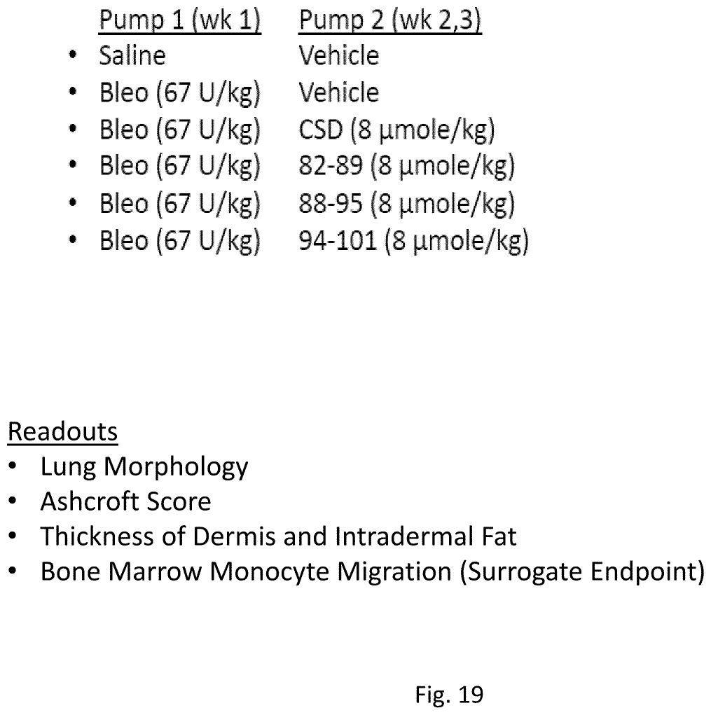

Experiments were conducted to examine the effect of unmodified CSD subdomains on lung and skin fibrosis. FIG. 19 provides details on the experimental design for experiments demonstrating suppression of lung and skin fibrosis by unmodifed CSD subdomains. Pumps containing bleomycin or saline vehicle were implanted subcutaneously into mice. After one week, the pumps had emptied and were replaced with pumps designed to empty over two weeks containing the indicated treatments (n=6 per group). After these two weeks, the mice were sacrificed.

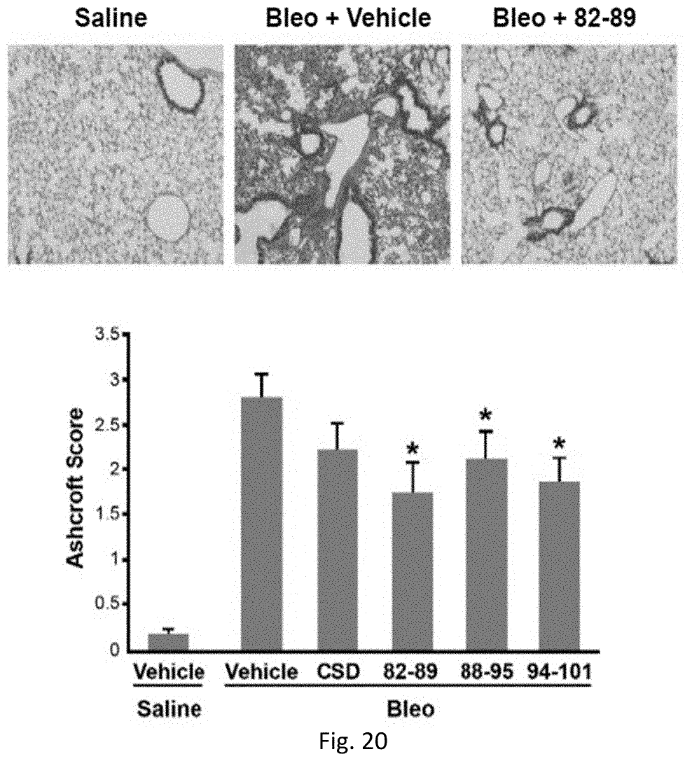

FIG. 20 depicts data demonstrating the massive fibrosis caused by bleomycin and its suppression by the 82-89, 88-95, and 94-101 subdomain peptides as read out in terms of Ashcroft score (n=6). *p<0.05 for Bleo+Veh vs Bleo+CSD subdomains

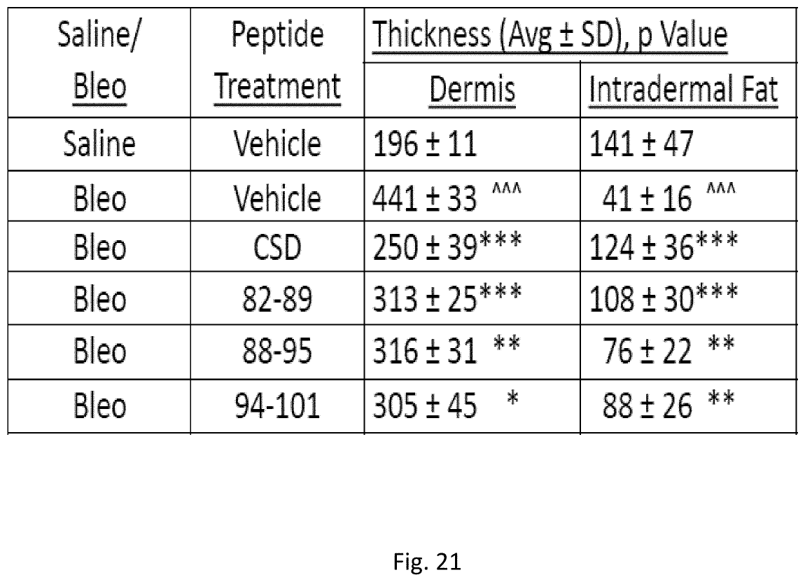

FIG. 21 demonstrates that there was suppression of dermal fibrosis and loss of intradermal fat by CSD and CSD subdomain peptides. Skin in the vicinity of the pump outlet was harvested for measurement of the thickness of the dermis and the intradermal fat. \hat{ }\hat{ }\hat{ }p<0.001 for Saline/Vehicle vs Bleo/Vehicle. ***p<0.001, **p<0.01, *p<0.05 for Bleo/Vehicle vs Bleo/Peptide Treatments.

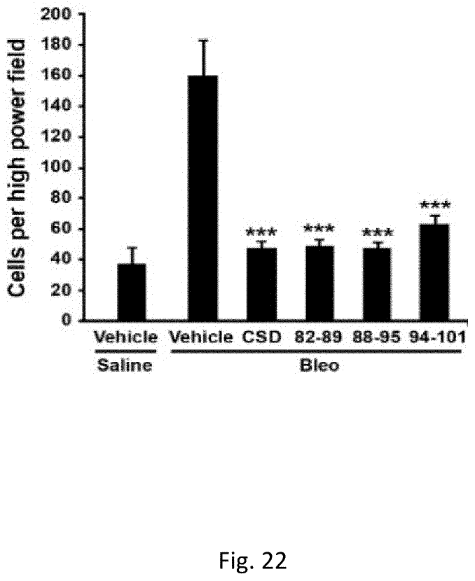

Recruitment of activated monocytes into damaged lung tissue contributes to the progression of fibrosis and is suppressed in vivo by CSD (FIG. 22). Bone marrow (BM) monocytes were isolated from control and bleomycin-treated mice. Significantly enhanced migration of BM monocytes was observed from bleomycin-treated mice compared to control mice. This enhanced migration is almost completely suppressed by treating the mice in vivo with each of the unmodified subdomains prior to harvesting the monocytes. ***p<0.001 for Bleo/Vehicle vs Bleo/Peptide Treatments.

In all of these readouts, all three subdomains of CSD were active. In general, 82-89 was the most active of the subdomains tested.

### Example 5: Suppression of Lung and Skin Fibrosis by a Modified, Water-Soluble Version of CSD

Experiments were also conducted to examine the effect of modified water-soluble versions of CSD subdomains on lung and skin fibrosis.

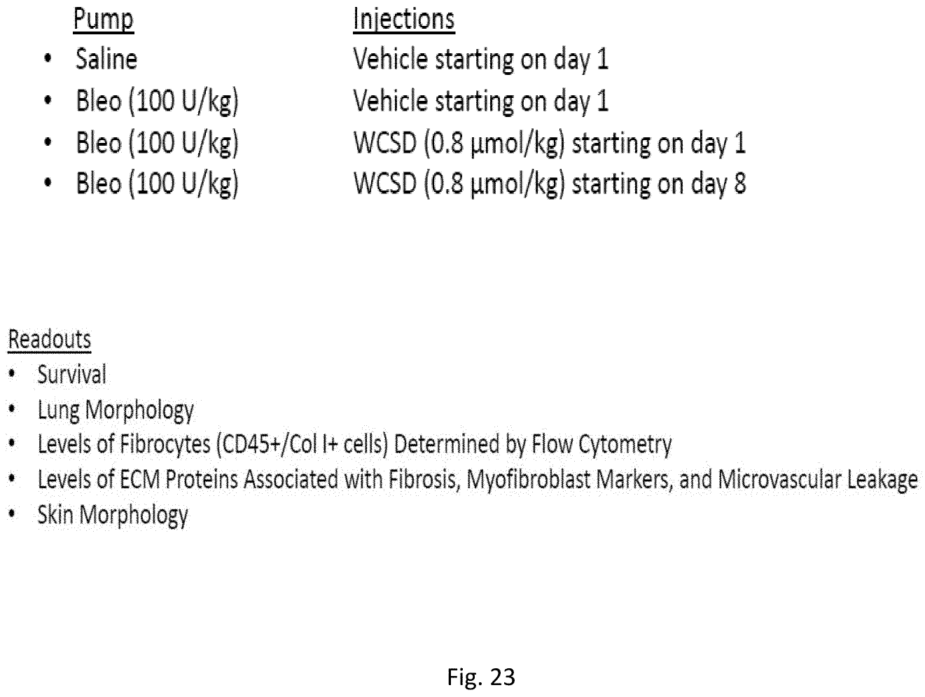

FIG. 23 provides details on the experimental design for experiments demonstrating suppression of lung and skin fibrosis by a modified, water-soluble version of CSD (WCSD; SEQ ID NO:5). Pumps containing bleomycin or saline vehicle were implanted subcutaneously into mice. After one week, the pumps had emptied. In addition, mice were injected i.p. daily as indicated, then sacrificed on day 22. All groups contained 4 mice.

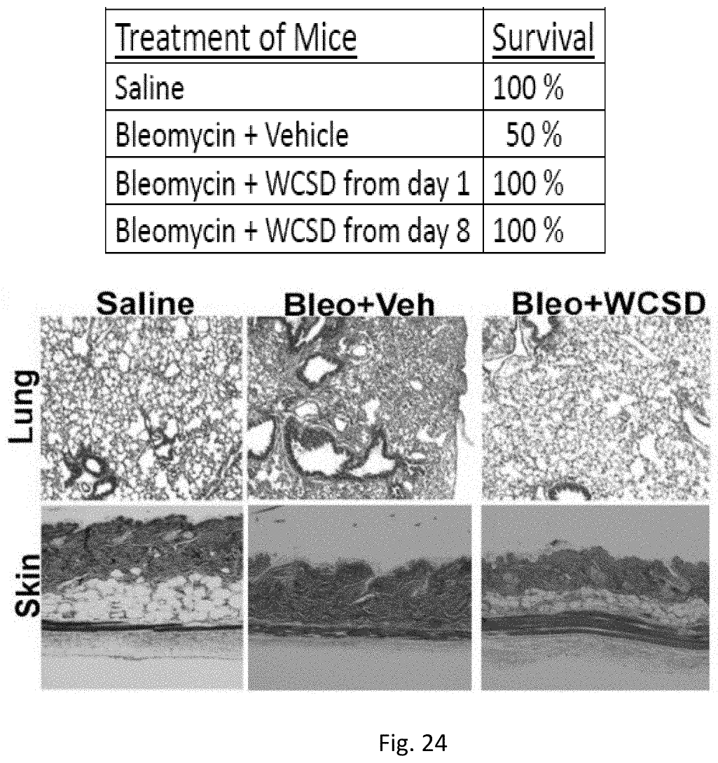

FIG. 24 provides survival and histology data demonstrating that the WCSD has an outstanding beneficial effect on survival. Massive lung fibrosis and inflammatory cell infiltration is caused by bleomycin and is suppressed by WCSD even when treatment is delayed until day 8. WCSD treatment beginning on day 8 also suppresses the almost complete loss of the transdermal adipose layer in the skin induced by bleomycin.

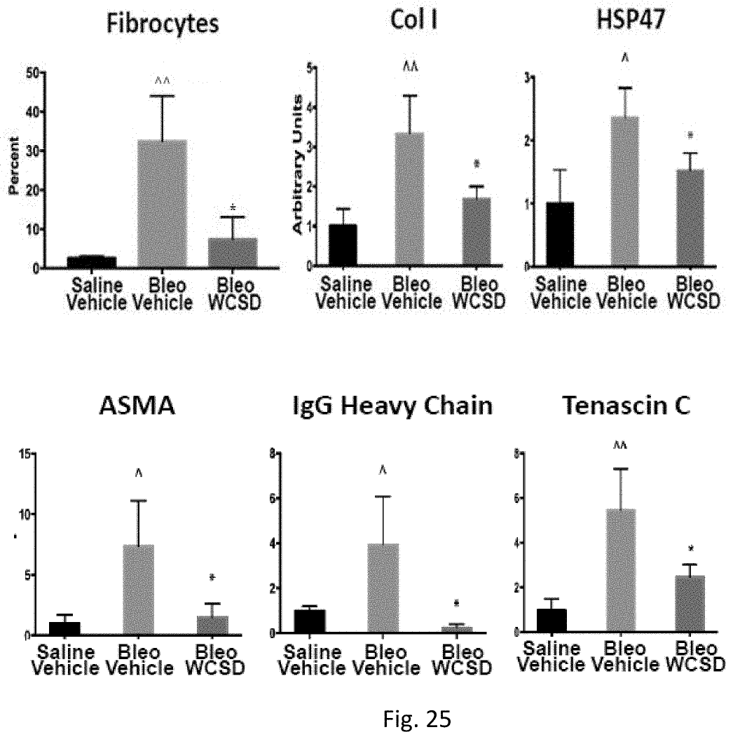

FIG. 25 demonstrates that WCSD suppresses bleomycin-induced lung fibrosis through its effects on fibrocytes, ECM proteins, myofibroblast markers, and microvascular leakage. Fibrocytes (CD45+/Col I+ cells) were quantified by Flow Cytometry. ECM proteins (Col I, Tenascin C), myofibroblast markers (HSP47, ASMA), and microvascular leakage (IgG Heavy Chain that remained in the tissue after perfusion) were quantified by Western blotting (n=4). Western blot data were quantified densitometrically following normalization to an actin loading control. The value in Saline treated mice was set to 1.0 Arbitrary Unit. \hat{ }\hat{ }p<0.01, \hat{ }p<0.05 for Saline/Vehicle vs Bleo/Vehicle; *p<0.05 for Bleo/Vehicle vs Bleo/WCSD.

All of these parameters were greatly increased by bleomycin-treatment and almost completely suppressed by WCSD down to the levels in control, saline-treated mice.

### Example 6: Inhibition of Tumor Growth by WCSD

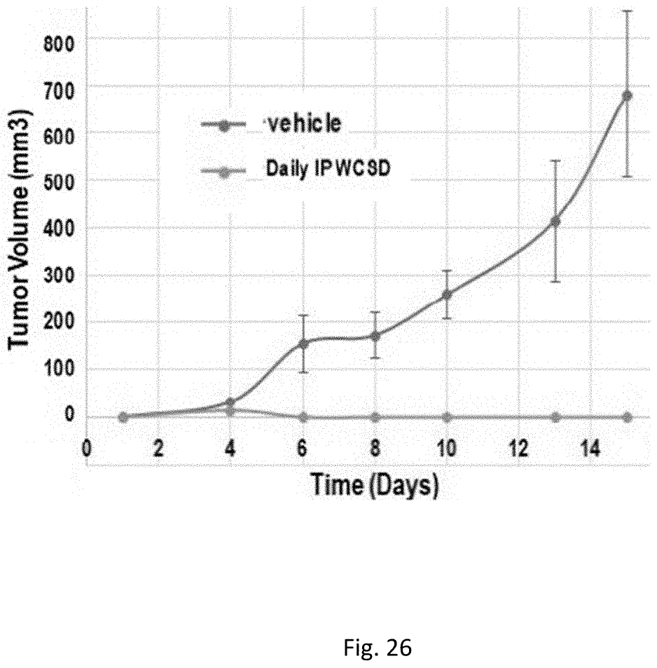

The function of cells in the tumor stroma (cancer-associated fibroblasts, endothelial cells) is modified by the tumor to promote its growth. Multiple groups have observed that cells in the tumor stroma in both humans and mice are deficient in caveolin-1. Therefore, the ability of WCSD (SEQ ID NO:5) to inhibit tumor growth was tested in syngeneic mice injected orthotopically with Met1 breast cancer cells. Mice received daily i.p. injections of vehicle or of WCSD (0.8 μmol/kg) starting the day after tumor cell injection (n=6 mice/group) resulting in 100% inhibition of tumor growth (FIG. 26).

### Example 7: Inhibition of Purified Kinases by Water-Soluble Versions of CSD

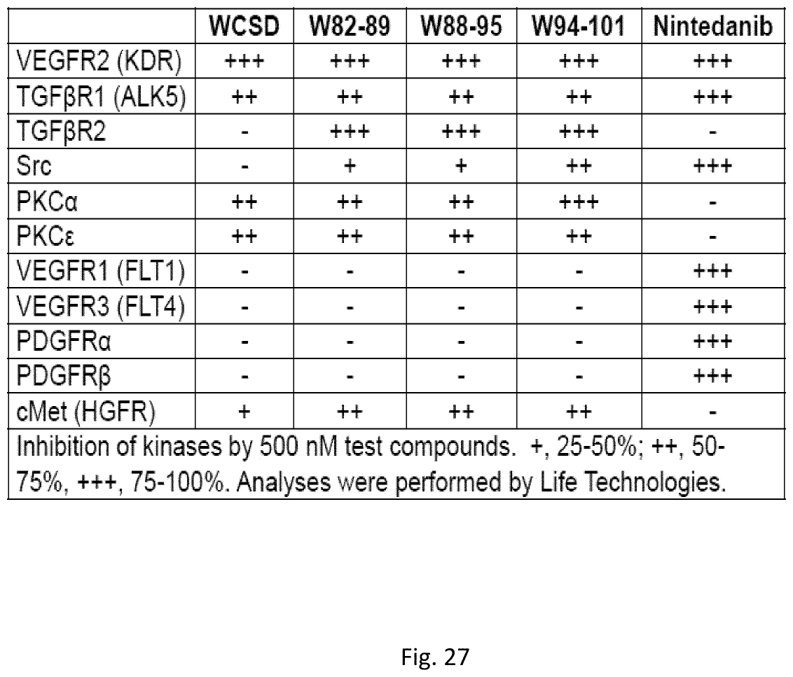

Water-soluble versions of CSD peptides inhibit TGFβR2, PKCα, PKCε, and cMet while nintedanib has no effect on these kinases (FIG. 27). Conversely, nintedanib inhibits VEGFR1, VEGFR3, PDGFRα, and PDGFRβ while the water-soluble versions of CSD show no direct inhibition. Both nintedanib and the water-soluble versions of CSD peptides inhibit VEGFR2, TGFβR1, and Src; however, nintedanib worked at much lower concentrations. These observations, along with the fact that the water soluble CSD peptides work in vivo at >100-fold lower concentration, indicate that nintedanib and the water soluble CSD peptides must have distinct mechanisms of action. This supports the idea that the water soluble CSD candidates may have much less severe side effects than are known to occur for nintedanib.

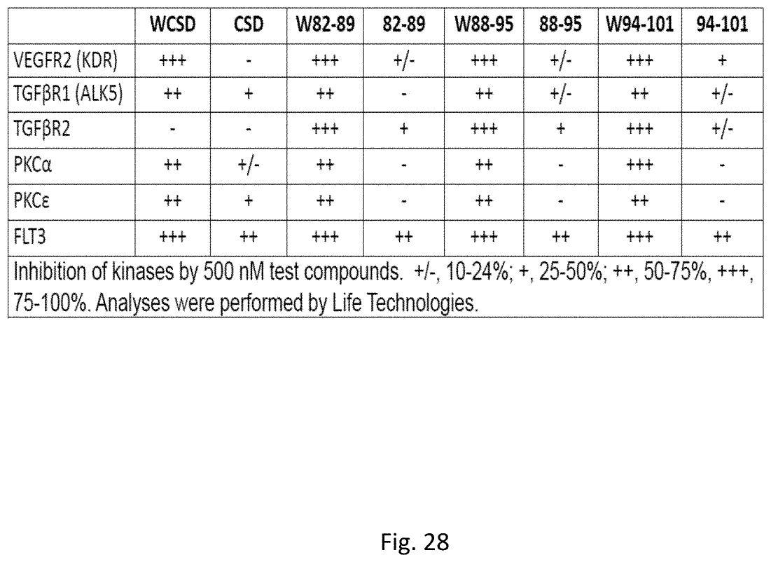

The modified, water soluble CSD peptides are more active as kinase inhibitors than their parental, unmodified forms (FIG. 28).

### Example 8: Optimization of Delivery

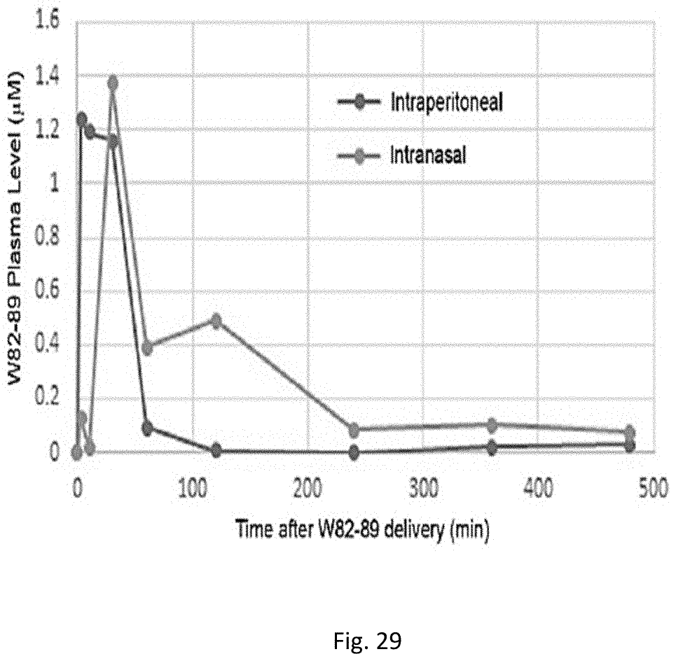

Experiments were conducted to evaluate different routes of delivery of water soluble CSD peptides. FIG. 29 demonstrates that intranasal (i.n.)

administration of the water soluble CSD peptides is a promising route of delivery. Two mice received a single i.p. injection of 0.8 μmol/kg fluoresceinated W82-89 in 100 μl PBS. Two mice received fluoresceinated W82-89 i.n. (4 μmol/kg in 30 μl PBS (15 μl per nostril)). Using calibrated capillary tubes, 50 μl of blood was collected from the maxillary vein from one mouse from each pair at 3 min, 30 min, 2 h, and 6 h; and from the other mouse from each pair at 10 min, 1 h, 4 h, and 8 h. Blood was immediately diluted with 200 μl PBS/10 mM EDTA, and centrifuged to harvest plasma. The level of W82-89 in the plasma was determined using a fluorescent plate reader.

Intranasal (i.n.) delivery as described provided a peak serum level similar to i.p. delivery. Moreover, i.n. delivery resulted in the prolonged presence of W82-89 in the plasma with an Area Under the Curve for i.n. delivery being >1.5-fold higher than for i.p. dose. These results suggest that i.n. delivery, besides being preferred by patients, may be an effective approach to delivering W82-89.

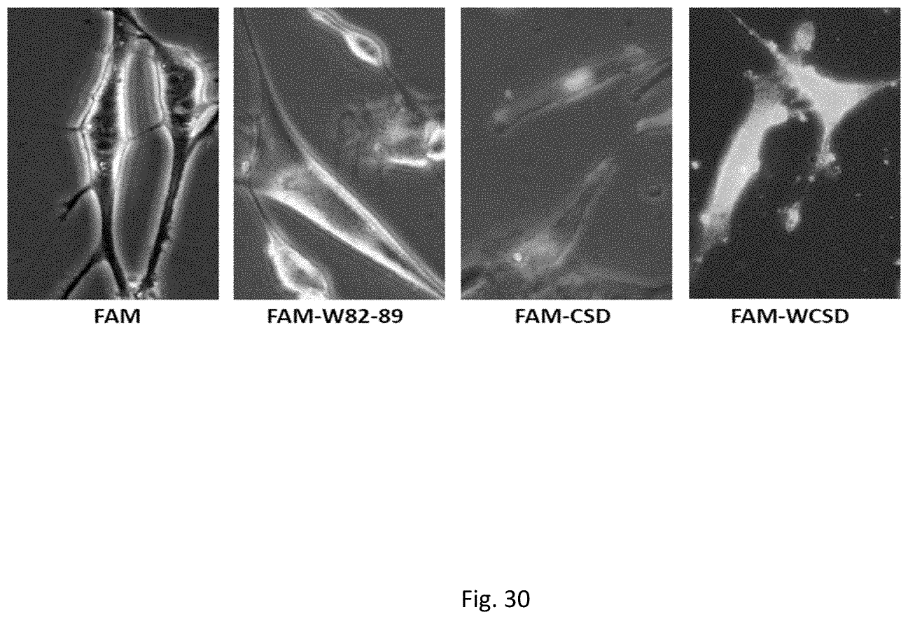

Primary lung fibroblasts were cultured for 4 hours in complete medium (DMEM/10% serum) supplemented with 5 μM of the indicated peptides synthesized with the fluorescent dye FAM at the N-terminus or with free FAM. While free FAM was not taken up by cells, FAM-labeled W82-89 and FAM-labeled CSD were taken up and FAM-labeled WCSD was taken up to a much greater extent (FIG. 30). This indicates that the modification that makes CSD water soluble also greatly improves its uptake by cells.

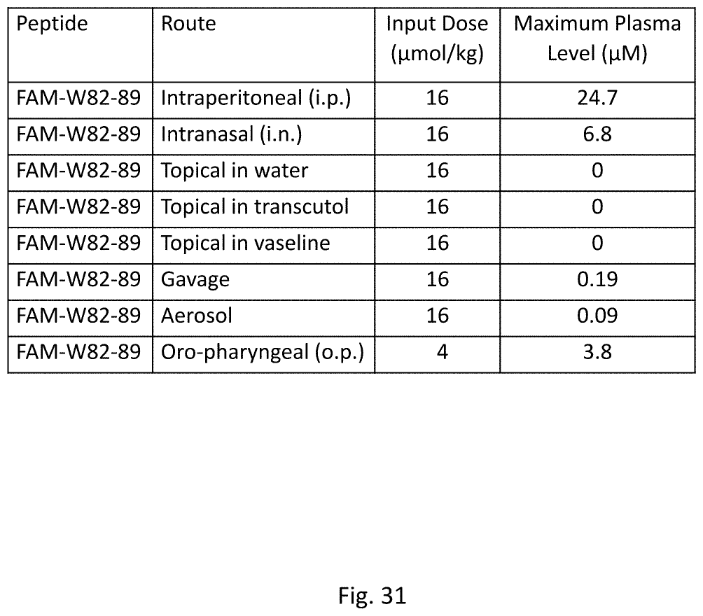

Following a single dose of fluoresceinated W82-89 delivered by the indicated routes (FIG. 31), blood was collected from the maxillary vein at intervals and the level of W82-89 in plasma derived from the blood was determined using a fluorescent plate reader. Note that following i.p. delivery, a high level of fluorescent peptide was present in the plasma. However, almost no fluorescent peptide was present in the plasma following topical delivery, gavage, or aerosol. The i.n. and o.p. routes, a favorable routes for treating patients, gave a relatively high level of fluorescent peptide in the plasma. It is likely that the i.n. and o.p. routes involve uptake via the lungs.

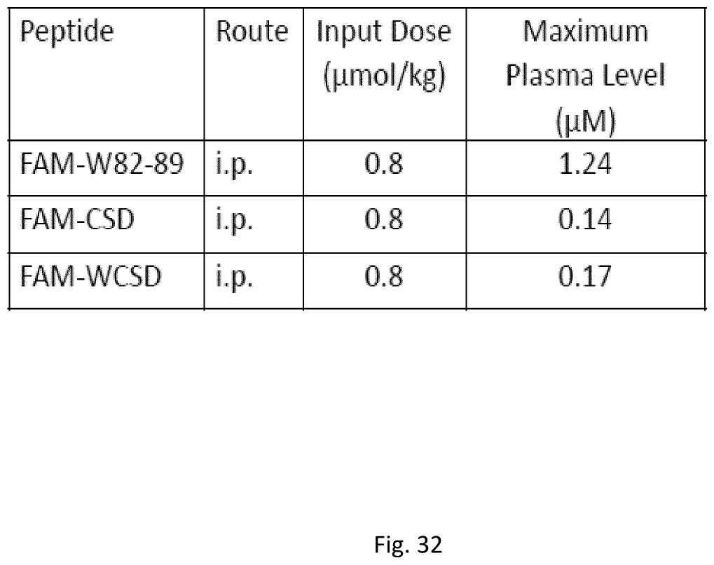

Following a single i.p. dose of the indicated fluorescent peptides, blood was collected from the maxillary vein at intervals and the levels of the peptides in plasma derived from the blood was determined using a fluorescent plate reader. The uptake of W82-89 was much greater than the uptake of CSD or WCSD (FIG. 32).

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

1. A method of treating or preventing microvascular leakage or a disease or disorder associated therewith, kidney disease, heart disease, or an age-related disease or disorder in a subject, the method comprising administering to a subject in need thereof an effective amount of a composition comprising a CSD domain peptide, or fragment or variant thereof, or a nucleic acid molecule encoding a CSD domain peptide, or fragment or variant thereof, wherein the CSD domain peptide comprises an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7 and SEQ ID NO:8.

2. The method of claim 1, wherein the method is a method of treating or preventing:
(a) an age-related disease or disorder selected from the group consisting of atherosclerosis, cardiovascular disease, microvascular leakage, cancer, arthritis, cataracts, osteoporosis, Alzheimer's disease and related neurodegenerative diseases and hypertension;
(b) a kidney disease selected from the group consisting of renal inflammatory injury, kidney dysfunction, chronic kidney failure, and hypertension;
(c) a heart disease selected from the group consisting of cardiac hypertrophy, atherosclerosis, cardiomyopathy, stroke, and hypertension; or
(d) a disease or disorder associated with microvascular leakage selected from the group consisting of congestive heart failure, scleroderma and interstitial lung diseases in general, asthma, kidney failure, neurodegenerative diseases including Alzheimer's disease and vascular dementia, cancer, venous thrombosis, diabetes and complications of diabetes, sepsis, and acute respiratory distress syndrome (ARDS).

3. (canceled)

4. (canceled)

5. (canceled)

6. A method of treating or preventing a disease or disorder in a subject, the method comprising administering to a subject in need thereof an effective amount of a composition comprising a CSD domain peptide, or fragment or variant thereof, or a nucleic acid molecule encoding a CSD domain peptide, or fragment or variant thereof, wherein the CSD domain peptide comprises an amino acid sequence selected from the group consisting of SEQ ID NO:5, SEQ ID NO:6, SEQ ID NO:7 and SEQ ID NO:8.

7. The method of claim 6, wherein the disease or disorder is:
(a) an age-related disease or disorder selected from the group consisting of atherosclerosis, cardiovascular disease, microvascular leakage, cancer, arthritis, cataracts, osteoporosis, Alzheimer's disease and related neurodegenerative diseases and hypertension;
(b) fibrosis or a fibrosis-related disease or disorder;
(c) microvascular leakage or a microvascular-leakage related disease or disorder;
(d) kidney disease; or
(e) heart disease.

8. (canceled)

9. (canceled)

10. The method of claim 7, wherein the microvascular leakage-related disease or disorder is selected from the group consisting of congestive heart failure, scleroderma and interstitial lung diseases in general, asthma, kidney failure, neurodegenerative diseases including Alzheimer's disease and vascular dementia, cancer, venous thrombosis, diabetes and complications of diabetes, sepsis, or acute respiratory distress syndrome (ARDS).

11. (canceled)

12. The method of claim 7, wherein the kidney disease is renal inflammatory injury, kidney dysfunction, chronic kidney failure, or hypertension.

13. (canceled)

14. The method of claim 7, wherein the heart disease is cardiac hypertrophy, atherosclerosis, cardiomyopathy, stroke, or hypertension.

15. A modified CSD domain peptide comprising an amino acid sequence selected from the group consisting of SEQ ID NO:5, SEQ ID NO:6, SEQ ID NO:7 and SEQ ID NO:8, or a fragment or variant thereof.

16. A composition comprising a modified CSD domain peptide of claim 15.

17. The composition of claim 16 further comprising a pharmaceutically acceptable carrier.

18. The composition of claim 16, wherein the composition treats or prevents a disease or disorder in a subject.

19. The composition of claim 18, wherein the disease or disorder is:
(a) an age-related disease or disorder selected from the group consisting of atherosclerosis, cardiovascular disease, kidney disease, microvascular leakage, cancer, arthritis, cataracts, osteoporosis, AD and related neurodegenerative diseases, complications of diabetes, and hypertension;
(b) fibrosis or a fibrosis-related disease or disorder;
(c) microvascular leakage or a microvascular-leakage related disease or disorder
(d) kidney disease; or
(e) heart disease.

20. (canceled)

21. (canceled)

22. The composition of claim 19, wherein the microvascular-leakage related disease or disorder is selected from the group consisting of congestive heart failure, scleroderma and interstitial lung diseases in general, asthma, kidney failure, neurodegenerative diseases including Alzheimer's disease and vascular dementia, cancer, venous thrombosis, diabetes and complications of diabetes, sepsis, and acute respiratory distress syndrome (ARDS).

23. (canceled)

24. The composition of claim 19, wherein the kidney disease is renal inflammatory injury, kidney dysfunction, chronic kidney failure, or hypertension.

25. (canceled)

26. The composition of claim 19, wherein the heart disease is cardiac hypertrophy, atherosclerosis, cardiomyopathy, stroke, or hypertension.

27. The composition of claim 16, wherein the composition is formulated for administration by a delivery route selected from the group consisting of intranasal, oro-pharyngeal and intraperitoneal.

28. A composition for treating or preventing microvascular leakage or a disease or disorder associated therewith, kidney disease, heart disease, or an age-related disease or disorder, comprising a CSD domain peptide comprising an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7 and SEQ ID NO:8, or a fragment or variant thereof.

29. The composition of claim 28, wherein the composition is used for treating or preventing:
(a) an age-related disease or disorder selected from the group consisting of atherosclerosis, cardiovascular disease, kidney disease, microvascular leakage, cancer, arthritis, cataracts, osteoporosis, AD and related neurodegenerative diseases, complications of diabetes, and hypertension;
(b) a kidney disease selected from the group consisting of renal inflammatory injury, kidney dysfunction, chronic kidney failure, and hypertension;
(c) a heart disease selected from the group consisting of cardiac hypertrophy, atherosclerosis, cardiomyopathy, stroke, and hypertension; or
(d) a microvascular leakage associated disease or disorder selected from the group consisting of congestive heart failure, scleroderma and interstitial lung diseases in general, asthma, kidney failure, neurodegenerative diseases including Alzheimer's disease and vascular dementia, cancer, venous thrombosis, diabetes and complications of diabetes, sepsis, or acute respiratory distress syndrome (ARDS).

30. (canceled)

31. (canceled)

32. (canceled)

33. The composition of claim 28, wherein the composition is formulated for administration by a delivery route selected from the group consisting of intranasal, oro-pharyngeal and intraperitoneal.
